(19) United States
(12) Patent Application Publication
(10) Pub. No.: US 2020/0063210 A1
(43) Pub. Date: Feb. 27, 2020

(54) Gene Expression Signatures Associated with Patient Response to Acute Myeloid Leukemia Treatment and Use Thereof for Predicting Response to Therapy

(71) Applicant: WAKE FOREST UNIVERSITY HEALTH SCIENCES, Winston-Salem, NC (US)

(72) Inventors: Timothy S. Pardee, Winston-Salem, NC (US); Lance D. Miller, Winston-Salem, NC (US)

(21) Appl. No.: 16/466,663

(22) PCT Filed: Dec. 5, 2017
(86) PCT No.: PCT/US2017/064658

§ 371 Date: Jun. 5, 2019

Related U.S. Application Data
(60) Provisional application No. 62/430,280, filed on Dec. 5, 2016.

(51) Int. Cl. C12Q 1/6886; G06F 17/18; G16H 50/30; G16B 25/10

(52) U.S. Cl. CPC C12Q 1/6886; G06F 17/18; G16H 50/30; G01N 2800/7028; C12Q 2600/106; C12Q 2600/158; G16B 25/10

(57) ABSTRACT

Compositions and methods are disclosed for identifying patients for treatment of hematological malignancy.

This invention was made with government support under Grant Numbers NCI 1K08CA169809-05 and 1R01CA197991-01A1. The government has certain rights in the invention.

### FIELD OF THE INVENTION

This invention relates to the fields of oncology and medicine. More specifically, the invention provides biomarkers and methods of use thereof which aid the clinician in identifying those patients most likely to benefit from treatment and monitoring response to treatment. The markers disclosed herein are also useful in assays to identify therapeutic agents useful for the treatment of malignancy.

### BACKGROUND OF THE INVENTION

Several publications and patent documents are cited throughout the specification in order to describe the state of the art to which this invention pertains. Each of these citations is incorporated herein by reference as though set forth in full.

Acute myeloid leukemia (AML) is an aggressive malignancy that leads to the accumulation of immature myeloid precursors, resulting in progressive marrow failure and death (1). It affects approximately 18,000 people per year in the United States; despite decades of research, the overall 5-year survival remains a disappointing 30-40% (2). The most common current treatment is intensive induction chemotherapy followed by consolidation using chemotherapy or a stem cell transplant (2). Induction therapy utilizes a combination of cytarabine and an anthracycline given over 7 and 3 days, respectively in the “7+3” regimen (3). This regimen was first reported in 1976 and has not substantially changed since (4). Although this regimen results in a 60 to 80% remission rate, the long-term survival rate is between 20 and 40% (5). This is driven by the fact that over 50% of patients will experience a relapse, and most of them will die from AML within a year (6).

After relapse, 20-50% of patients achieve a second remission in response to salvage therapy (7, 8) and median survival is under 6 months in most studies (9, 10). A recent study compared the efficacy of the novel agent, elacytarabine, versus the investigator's choice of one of seven commonly used salvage regimens. The overall remission rate was only 22%, and the median survival was 3.4 months (11). Despite intense efforts, the basic cellular mechanisms behind resistance in AML are not well understood.

Recurrent chromosomal alterations are highly prognostic in AML and can be used to place patients into good, intermediate or poor risk groups (12). Poor risk patients have only a 32% remission rate, a 92% 5 year relapse rate and a dismal 5% 5 year survival (12). These numbers are worse in relapse where in one study relapsed or refractory poor risk AML patients had a 19% response rate a median survival of just 2.8 months (8). Loss or mutation of p53 is common in poor risk patients and carries a dismal prognosis (13). There is a clear need for improved therapies for AML in general and for poor risk patients in particular.

### SUMMARY OF THE INVENTION

In accordance with one aspect, the present invention provides a method for detecting a collection of differentially expressed genes which indicate a subject's sensitivity to treatment with CPI-613. The method involves the following steps: a) obtaining a biological sample (e.g., cerebral spinal fluid, bone marrow, mononuclear cell isolate from bone marrow, or peripheral blood cells) from a subject and b) detecting in the biological sample the expression level of at least three genes selected from the group consisting of RP11-164H13.1, FCRLA, KLHL14, IGKV2D-29, MS4A1, DSP, COL19A1, IGKV1-8, PCDH9, TCL1A, RP11-693J15.5, IGHV3-53, IGHV3-11, FCRL2, PTPRK, BANK1, SLC16A9, and CD79. In a particular embodiment, the following three genes are detected: RP11-164H13.1, FCRLA, and KLHL14. In another embodiment, subjects that are likely to be responders or non-responders to CPI-613 treatment are segregated by hierarchical cluster analysis based on expression of TCL1A, MS4A1, FCRL2, PTPRK, CD79A, BANK1, RP11-164H13.1, and RP11-693J15.5.

In accordance with another aspect, the present invention provides a method for detecting the differential expression of RP11-164H13.1 in a biological sample obtained from a subject which indicates the subject's sensitivity to treatment with CPI-613. The method includes the following steps: a) obtaining a biological sample (e.g., cerebral spinal fluid, bone marrow, mononuclear cells isolated from bone marrow, or peripheral blood cells) from the subject and b) detecting the expression level of RP11-164H13.1 in the biological sample.

In accordance with another aspect, the present invention provides a method of identifying and treating a subject likely to respond to CPI-613 treatment of AML. The method involves the following steps: a) obtaining a biological sample from the subject; b) detecting expression levels of at least three genes selected from the group consisting of RP11-164H13.1, FCRLA, KLHL14, IGKV2D-29, MS4A1, DSP, COL19A1, IGKV1-8, PCDH9, TCL1A, RP11-693J15.5, IGHV3-53, IGHV3-11, FCRL2, PTPRK, BANK1, SLC16A9, and CD79, where elevated levels indicate sensitivity to CPI-613; and d) administering to the subject a treatment that includes CPI-613 and, optionally, at least one other chemotherapeutic agent. In a particular embodiment, the subject is treated with CPI-613, cytarabine, and mitoxantrone.

In a further aspect, the present invention provides a method of assessing whether a subject is likely to respond to CPI-613 treatment of AML by detecting the above identified markers with antibodies. The method includes the following steps: a) obtaining a biological sample from a subject; b) contacting the sample with an antibody immunologically specific for at least three markers selected from the group consisting of RP11-164H13.1, FCRLA, KLHL14, IGKV2D-29, MS4A1, DSP, COL19A1, IGKV1-8, PCDH9, TCL1A, RP11-693J15.5, IGHV3-53, IGHV3-11, FCRL2, PTPRK, BANK1, SLC16A9, and CD79; and c) detecting immune complex formation between the antibody and marker, where the presence of the immune complexes is predictive of sensitivity to CPI-613 treatment. In a particular embodiment the antibody or antibodies used for detecting the markers are labeled for use in immunohistochemistry, immunofluorescence imaging, or flow cytometry.

In certain embodiments, gene expression levels are measured using RNA-seq, RT-qPCR, or NanoString's nCounter technology.

In accordance with certain aspects of the invention, the subject has relapsed or refractory AML. In another embodiment, the method is performed after treatment with CPI-613, in order to assess the patient's response to therapy. In particular embodiments, the subject has been additionally identified as having p53 loss or mutation.

### BRIEF DESCRIPTION OF THE DRAWINGS

FIG. 1. CPI-613 is a novel lipoate derivative that inhibits the TCA cycle. The structure of lipoate and CPI-613 are shown on the right. On the left is a simplified schematic of carbon metabolism with the CPI-613 targets shown in red.

FIGS. 2A-2B. Chemotherapy induces mitochondrial metabolism in AML cells. K562 cells (FIG. 2A) and OCI-AML3 (FIG. 2B) and were treated with Ara-C for 16 hours and oxygen consumption rate (OCR) was assessed. Shown is the average OCR from 3 independent experiments each done in triplicate. P value for OCI-AML3 is 0.0387 and for K562 is <0.0001.

FIGS. 3A-3B. TCA cycle contributes to chemotherapy resistance. PDH deleted AML cells were generated by infecting sgRNAs against PDH E1a into constitutive expressing Cas9 MFL2 cells. Cells were incubated with the Doxorubicin (FIG. 3A) or Ara-C (FIG. 3B) for 72 hours and viability determined.

FIG. 4. CPI-613 acts synergistically with doxorubicin in vivo. Balb/c mice were injected with Baf3-p210 cells and on day 3 treated with saline (control), Doxorubicin at 3 mg/kg or CPI at 250 mg/kg plus doxorubicin. Dox=Doxorubicin, CPI=CPI-613. P value shown is for Dox vs CPI-Dox.

FIG. 5. Phase I Clinical Trial Schema.

FIG. 6. Patient characteristics.

FIG. 7. CPI-613 clinical activity summary.

FIGS. 8A-8B. Survival compared to historical cohort. CPI-HAM survival in the evaluable population compared to a historical cohort. (FIG. 8A) Kaplan-Meier curve of overall survival of elderly AML patients treated with high dose cytarabine, mitoxantrone, and CPI-613 (CPI-HAM) or a historical cohort of poor risk relapsed or refractory patients treated with high dose cytarabine, mitoxantrone, and asparaginase (HAMA). (FIG. 8B) Kaplan-Meier curve of overall survival of poor risk, relapsed or refractory AML patients treated with high dose cytarabine, mitoxantrone, and CPI-613 (CPI-HAM) or a historical cohort of poor risk relapsed or refractory patients treated with high dose cytarabine, mitoxantrone, and asparaginase (HAMA). Kaplan-Meier estimates were compared by log-rank test.

FIGS. 9A-9C. AMPK Contributes to Resistance to CPI-613. (FIG. 9A) CPI-613 induces AMPK phosphorylation. Blood samples were taken on day one of treatment at the indicated time points following infusion of CPI-613. Red blood cells were lysed, and mononuclear cells were isolated. Extracts were prepared and blotted for phosphorylated AMPK. Both patients had circulating blasts at the time of sampling. Actin was used as a loading control. (FIG. 9B) Competition assay. Cas9 expressing MFL2 cells were partially infected with an sgRNA vector targeting AMPK tagged with GFP. Cells were incubated with the indicated drug for 72 hours and the % GFP+determined in the viable population. (FIG. 9C) Viability assay. Clones of Cas9 expressing MFL2 cells with sgRNAs targeting AMPK or ROSA26 were incubated with the indicated drug for 72 hours and viability determined.

FIGS. 10A-10C. CPI-613 induces chemotherapy sensitivity in p53 suppressed AML cells. (FIG. 10A) Murine AML cells (M1p5) expressing an shRNA against p53 (p53 shRNA) or vector control (GFP Control) were exposed to the indicated amount of CPI-613 (in μmoles). After 72 hours viability was determined and compared to untreated cells. (FIG. 10B) Murine AML cells (M1p5) expressing an shRNA against p53 (p53 shRNA) or vector control (GFP Control) were exposed to the indicated amount of doxorubicin (in nmoles). After 72 hours viability was determined and compared to untreated cells. (FIG. 10C) Murine AML cells (M1p5) expressing an shRNA against p53 (p53 shRNA) or vector control (GFP Control) were exposed to 50 μM CPI-613 and 2.5, 5, or 10 nM Doxorubicin for 72 hours. Following exposure viability was assessed and compared to untreated cells.

FIG. 11. Gene expression analysis of bone marrow samples. Analysis for enrichment of gene ontologies revealed several significant biological categories for the genes overexpressed in responders, while none were identified for the non-responders. The responders showed significant enrichment for gene categories related to immune involvement. Heat map shows differentially expressed genes between responders (R) and nonresponders (NR).

FIGS. 12A-12D. RP11-164H13.1(TCL1A antisense) (FIG. 12A) Ensembl gene record for ENSG00000257275 (FIG. 12B) Transcript ID ENST00000556386.1 (FIG. 12C) Transcript ID ENST00000553445.5 (FIG. 12D) Transcript ID ENST00000547644.6

FIGS. 13A-13B. FCRLA (Fc receptor-like A) (FIG. 13A) Ensembl gene record for ENSG00000132185 (FIG. 13B) Transcript ID ENST00000367959.6

FIGS. 14A-14C. KLHL14 (Fc receptor-like A) (FIG. 14A) Ensembl gene record for ENSG00000257275 (FIGS. 14B and 14C) Transcript ID ENST00000359358.8

FIGS. 15A-15B. IGKV2D-29 (immunoglobulin kappa variable 2D-29) (FIG. 15A) Ensembl gene record for ENSG00000243264 (FIG. 15B) Transcript ID ENST00000491977.1

FIGS. 16A-16B. MS4A1 (membrane-spanning 4-domains, subfamily A, member 1) (FIG. 16A) Ensembl gene record for ENSG00000156738 (FIG. 16B) Transcript ID ENST00000534668.5

FIGS. 17A-17D. DSP (desmoplakin) (FIG. 17A) Ensembl gene record for ENSG00000096696 (FIGS. 17B-17D) Transcript ID ENST00000379802.7

FIGS. 18A-18C. COL19A1 (collagen, type XIX, alpha 1) (FIG. 18A) Ensembl gene record for ENSG00000082293 (FIGS. 18B-18C) Transcript ID ENST00000620364.4

FIGS. 19A-19B. IGKV1-8 (immunoglobulin kappa variable 1-8) (FIG. 19A) Ensembl gene record for ENSG00000240671 (FIG. 19B) Transcript ID ENST00000495489.1

FIGS. 20A-20C. PCDH9 (protocadherin 9) (FIG. 20A) Ensembl gene record for ENSG00000184226 (FIGS. 20B-20C) Transcript ID ENST00000544246.5

FIGS. 21A-21B. TCL1A (T-cell leukemia/lymphoma 1A) (FIG. 21A) Ensembl gene record for ENSG00000100721 (FIG. 21B) Transcript ID ENST00000402399.5

FIGS. 22A-22D. RP11-693J15.5 (lincRNA) (FIG. 22A) Ensembl gene record for ENSG00000205056 (FIGS. 22B-22D) Transcript ID ENST00000504409.3

FIGS. 23A-23B. IGHV3-53 (immunoglobulin heavy variable 3-53) (FIG. 23A) Ensembl gene record for ENSG00000211967 (FIG. 23B) Transcript ID ENST00000390627.3

FIGS. 24A-24B. IGHV3-11 (immunoglobulin heavy variable 3-11 (gene/pseudogene)) (FIG. 24A) Ensembl gene record for ENSG00000211941 (FIG. 24B) Transcript ID ENST00000390601.3

FIGS. 25A-25B. FCRL2 (Fc receptor-like 2) (FIG. 25A) Ensembl gene record for ENSG00000132704 (FIG. 25B) Transcript ID ENST00000361516.7

FIGS. 26A-26C. PTPRK (protein tyrosine phosphatase, receptor type, K) (FIG. 26A) Ensembl gene record for ENSG00000152894 (FIGS. 26B-26C) Transcript ID ENST00000368226.8

FIGS. 27A-27B. BANK1 (B cell scaffold protein with ankyrin repeats 1) (FIG. 27A) Ensembl gene record for ENSG00000153064 (FIG. 27B) Transcript ID ENST00000322953.8

FIGS. 28A-28C. SLC16A9 (solute carrier family 16, member 9) (FIG. 28A) Ensembl gene record for ENSG00000165449 (FIGS. 28B-28C) Transcript ID ENST00000395348.7

FIGS. 29A-29B. CD79A (CD79a molecule, immunoglobulin-associated alpha) (FIG. 29A) Ensembl gene record for ENSG00000105369 (FIG. 29B) Transcript ID ENST00000221972.7

### DETAILED DESCRIPTION OF THE INVENTION

AML is an aggressive malignancy with poor outcomes. Its treatment has not changed for four decades, and most patients will die of therapy-resistant disease. The presence of recurrent chromosomal alterations is highly prognostic in AML. Patients can be divided into good, intermediate or poor risk groups based on the karyotype of the tumor. Poor risk patients have significantly lower remission rates, higher relapse rates and shorter overall survival. The five year survival for poor risk AML patients is less than 10%. There is a desperate need for novel approaches for this group of patients.

Altered metabolism is a hallmark of cancer cells, including AML. Since the seminal observations of Otto Warburg it has been known that solid tumor cells have highly altered cellular metabolism. Tumor cells preferentially use glycolysis, even in the presence of oxygen, suppress oxidative phosphorylation, and employ tricarboxylic acid (TCA) cycle intermediates as biosynthetic precursors (reviewed in (14, 15)). This was initially thought to reflect some defect in tumor cell mitochondria but has now been shown to be the consequence of a change in the role of mitochondria from energy production to biosynthetic precursor generation. There is now growing evidence that this same phenomenon occurs in AML cells. Human AML cell lines exhibit the changes characteristic of altered glycolytic metabolism (16, 17), and the level of glycolysis is prognostic (18). Consistent with these data, AML cells are sensitive to glycolysis inhibitors (19) and extra-medullary AML is highly PET avid. Recently, increased levels of pyruvate and 2-oxatogluterate were reported in the serum of AML patients consistent with enhanced glucose metabolism (20) and the levels of these metabolites were prognostic (20). These data demonstrate that like solid tumors, AML utilizes altered metabolism.

Oxidative phosphorylation is down-regulated in most tumor cells; however, recent studies suggest it has a role in therapy response. Inhibition of mitochondrial metabolism at the level of the electron transport chain (ETC) enhances the effect of cisplatin and docetaxel in lung and prostate cancer cell lines (21). In patients with esophageal cancer, reduced expression of ETC members was associated with an improved response to cisplatin, and cell lines could be made more sensitive by their knockdown (22). In several cancers, therapy-resistant cells show reduced mitochondrial membrane potential, over-expression of the mitochondrial uncoupling protein UCP2, and utilization of fatty acid oxidation (FAO) (23). AML cells when co-cultured with bone marrow stromal cells become resistant to therapy, up-regulate UCP2, increase FAO and display decreased membrane potential (17). Additionally, the electron transport chain complex I inhibitor, metformin, induces apoptosis in AML cell lines and reduced colony formation in primary patient samples (24).

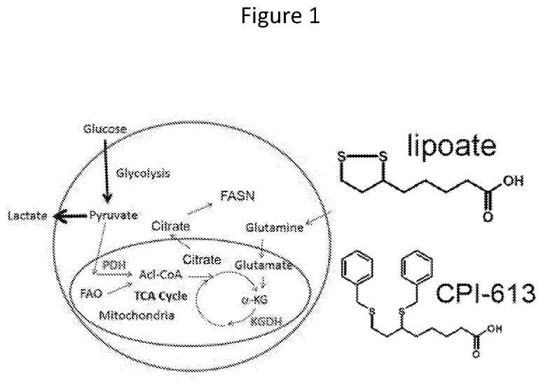

CPI-613 is a novel lipoate derivative that has shown extensive anticancer effects in preclinical studies (25, 26 and FIG. 1). It inhibits pyruvate dehydrogenase (PDH) via activation of pyruvate dehydrogenase kinase-induced phosphorylation. In contrast, its inhibitory action on α-ketogluterate dehydrogenase (KGDH) involves a novel redox-sensing mechanism that results in the glutathionylation and inactivation of the enzyme. This simultaneous inhibition of PDH and KGDH blocks entry of either glucose or glutamine-derived carbons into the TCA cycle, effectively preventing mitochondrial carbon metabolism and oxidative phosphorylation.

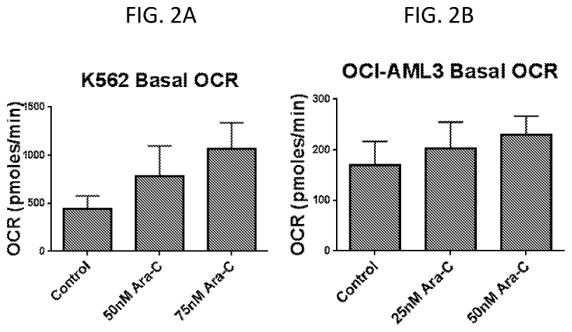

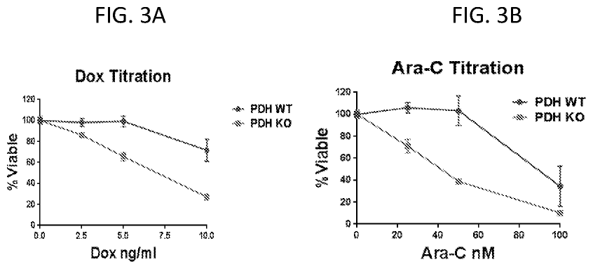

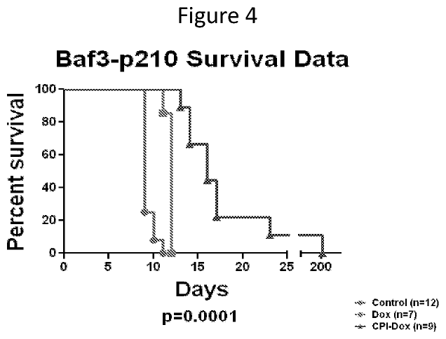

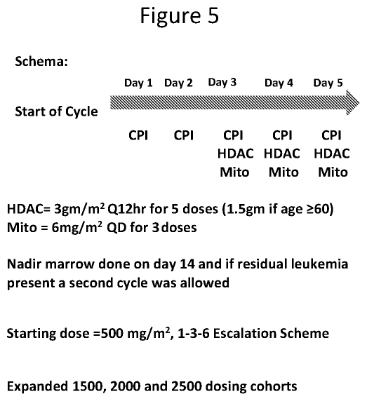

Mitochondrial inhibition by metformin or CPI-613 impairs AML cell growth in multiple human and murine cell lines and that the two together are synergistic in vitro and in vivo (FIG. 4). Additionally, when AML cells are exposed to chemotherapy they increase their mitochondrial respiration (FIGS. 2A and 2B) consistent with increased oxidative phosphorylation in response to DNA damage. Our data also demonstrate that the TCA cycle contributes to resistance to therapeutic agents (FIGS. 3A and 3B). In sum, mitochondrial metabolism is necessary for AML cell survival and is involved in resistance.

In a single agent phase one study, CPI-613 was active in several patients with advanced myeloid malignancies including a patient with refractory AML (27). This patient had failed several chemotherapy regimens as well as both hypomethylating agents prior to enrollment on trial. After two cycles of therapy the bone marrow was cleared of leukemia and the patient went on to a stem cell transplant. The patient is still alive and free of leukemia. Five additional patients had objective responses for an overall response rate of 29% in a heavily pre-treated cohort of patients. To our knowledge, this is the first report of a TCA cycle inhibitor that has activity in patients with hematologic malignances.

The following definitions are provided to facilitate an understanding of the present invention.

For purposes of the present invention, “a” or “an” entity refers to one or more of that entity; for example, “a cDNA” refers to one or more cDNA or at least one cDNA. As such, the terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds. According to the present invention, an isolated, or biologically pure molecule is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

A “sensitivity marker” is a marker (or biomarker) which is associated differential sensitivity to a treatment, for example a treatment that includes CPI-613. Such markers may include, but are not limited to, nucleic acids, proteins encoded thereby, or other small molecules. These markers can be used to advantage to identify those patients likely to respond to therapy from those that are unlikely to respond. They can also be targeted to modulate the response to therapy or used in screening assays to identify agents that have efficacy or act synergistically for the treatment and management of a hematological malignancy, such as AML.

Detection of the sensitivity markers can be carried out by standard histological and/or immuno-detection methods. In particular embodiments, the CPI-613 sensitivity markers can be detected by any means of polypeptide detection, or detection of the expression level of the polypeptides. For example, the polypeptide can be detected using any of antibody detection methods (e.g., immunofluorescent (IF) methods, flow cytometry, fluorescence activated cell sorting (FACS)), antigen retrieval and/or microarray detection methods can be used. A reagent that specifically binds to a marker polypeptide, e.g., an antibody, and antibody derivative, and an antibody fragment, can be used. Other detection techniques that can be used include, e.g., capture assays (e.g., ELISA), mass spectrometry (e.g., LCMS/MS), and/or polymerase chain reaction (e.g., RT-PCR). The markers can also be detected by systemic administration of a labeled form of an antibody to the markers followed by imaging. In another embodiment, the nucleic acid sample from the subject is evaluated by a nucleic acid detection technique as described herein.

In other embodiments, sensitivity markers are measured or detected by measuring mRNA expression. Numerous techniques such as qRT-PCR, Fluidigm, RNAseq (e.g. Illumina), Affymetrix gene profiling, the NanoString nCounter platform, or Nanopore sequencing (Oxford Nanopore Technologies) may be used by the person skilled in the art using their common general knowledge to measure the RNA levels and these may be calibrated against the IHC analysis to establish suitable scoring levels.

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon, polyacrylamide, dextran or agarose.

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the functional and novel characteristics of the sequence.

“Target nucleic acid” as used herein refers to a previously defined region of a nucleic acid present in a complex nucleic acid mixture wherein the defined wild-type region contains at least one known nucleotide variation which may or may not be associated with disease. The nucleic acid molecule may be isolated from a natural source by cDNA cloning or subtractive hybridization or synthesized manually. The nucleic acid molecule may be synthesized manually by the triester synthetic method or by using an automated DNA synthesizer.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form. By the use of the term “enriched” in reference to nucleic acid it is meant that the specific DNA or RNA sequence constitutes a significantly higher fraction (2-5 fold) of the total DNA or RNA present in the cells or solution of interest than in normal cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁻⁶-fold purification of the native message. Thus, purification of at least one order of magnitude, preferably two or three orders, and more preferably four or five orders of magnitude is expressly contemplated. Thus the term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. For example, specific hybridization can refer to a sequence which hybridizes to any IM sensitivity marker gene or nucleic acid, but does not hybridize to other nucleotides. Also polynucleotide which “specifically hybridizes” may hybridize only to a IM sensitivity marker shown in the Tables contained herein. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., Molecular Cloning, Cold Spring Harbor Laboratory (1989)):

Tₘ=81.5° C.+16.6 Log[Na+]+0.41(% G+C)−0.63(% formamide)−600/# bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ. of the hybrid. Wash conditions should be as stringent as possible for the degree of identity of the probe for the target. In general, wash conditions are selected to be approximately 12-20° C. below the Tₘ. of the hybrid. In regards to the nucleic acids of the current invention, a moderate stringency hybridization is defined as hybridization in 6×SSC, 5×Denhardt's solution, 0.5% SDS and 100m/m1 denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100m/m1 denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “oligonucleotide,” as used herein is defined as a nucleic acid molecule comprised of two or more ribo- or deoxyribonucleotides, preferably more than three. The exact size of the oligonucleotide will depend on various factors and on the particular application and use of the oligonucleotide. Oligonucleotides, which include probes and primers, can be any length from 3 nucleotides to the full length of the nucleic acid molecule, and explicitly include every possible number of contiguous nucleic acids from 3 through the full length of the polynucleotide. Preferably, oligonucleotides are at least about 10 nucleotides in length, more preferably at least 15 nucleotides in length, more preferably at least about 20 nucleotides in length.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25, 30, 50, 75 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

A “label” or a “detectable moiety” in reference to a nucleic acid, refers to a composition that, when linked with a nucleic acid, renders the nucleic acid detectable, for example, by spectroscopic, photochemical, biochemical, immunochemical, or chemical means. Exemplary labels include, but are not limited to, radioactive isotopes, magnetic beads, metallic beads, colloidal particles, fluorescent dyes, enzymes, biotin, digoxigenin, haptens, and the like. A “labeled nucleic acid or oligonucleotide probe” is generally one that is bound, either covalently, through a linker or a chemical bond, or noncovalently, through ionic bonds, van der Waals forces, electrostatic attractions, hydrophobic interactions, or hydrogen bonds, to a label such that the presence of the nucleic acid or probe can be detected by detecting the presence of the label bound to the nucleic acid or probe.

“Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytoxic/antitumor antibiotics, topoisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, RAF inhibitors, antisense oligonucleotides that that inhibit expression of genes implicated in abnormal cell proliferation or tumor growth. Chemotherapeutic agents useful in the treatment methods of the present invention include cytostatic and/or cytotoxic agents.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25, 30, 50, 75 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complimentary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation,” “transfection,” and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, PEG-fusion, and the like.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the IM sensitivity marker nucleic acid molecule such that the latter is transcribed into mRNA. Host cell machinery then translates mRNA into a polypeptide. The skilled artisan is aware of many other suitable promoter elements which can be used in the vectors of the invention.

Those skilled in the art will recognize that a nucleic acid vector can contain nucleic acid elements other than the promoter element and the IM sensitivity marker gene nucleic acid molecule. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

A “replicon” is any genetic element, for example, a plasmid, cosmid, bacmid, plastid, phage or virus, that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system,” “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The terms “recombinant organism,” or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. An organism can be as simple as one eukaryotic cell or as complex as a mammal. Therefore, the phrase “a recombinant organism” encompasses a recombinant cell, as well as eukaryotic and prokaryotic organism.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” or “specific binding” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form “immune complexes” with, i.e., to bind to, any antigens present. After this time, the sample-antibody composition, such as a cell suspension, tissue section, ELISA plate, dot blot or western blot, will generally be washed to remove any non-specifically bound antibody species, allowing only those molecules specifically bound within the primary immune complexes to be detected.

In general, the detection of immune complex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

“Sample” or “patient sample” or “biological sample” generally refers to a sample which may be tested for a particular molecule, preferably a sensitivity marker molecule. Samples may include but are not limited to cells, body fluids, including blood, bone marrow, serum, plasma, urine, saliva, tears, pleural fluid and the like.

The term “subject” refers to both animals and humans.

An “antibody” or “antibody molecule” is any immunoglobulin, including antibodies and fragments thereof, that binds to a specific antigen. The term includes polyclonal, monoclonal, chimeric, single domain (Dab) and bispecific antibodies. As used herein, antibody or antibody molecule contemplates recombinantly generated intact immunoglobulin molecules and immunologically active portions of an immunoglobulin molecule such as, without limitation: Fab, Fab′, F(ab′)₂, F(v), scFv, scFv₂, scFv-Fc, minibody, diabody, tetrabody, single variable domain (e.g., variable heavy domain, variable light domain), bispecific, Affibody® molecules (Affibody, Bromma, Sweden), and peptabodies (Terskikh et al. (1997) PNAS 94:1663-1668).

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

A “toxin” refers to a substance that inhibits or prevents the expression activity of cells, function of cells and/or causes destruction of cells. The term includes small molecule toxins or enzymatically active toxins of bacterial, fungal, plant or animal origin, including fragments and/or variants thereof which are effective to inhibit protein synthesis in a target cell. Examples of toxins include, but are not limited to: Pseudomonas exotoxin (PE) A, PE40, ricin, ricin A-chain, diphtheria toxin, abrin, abrin A chain, modeccin A chain, alpha-sarcin, gelonin, mitogellin, retstrictocin, phenomycin, enomycin, curicin, crotin, and calicheamicin. Antibodies may also be conjugated to such toxins, thereby forming an “immunotoxin” to facilitate targeting to cells.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), bulking substance (e.g., lactose, mannitol), excipient, auxilliary agent or vehicle with which an active agent of the present invention is administered. Pharmaceutically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. The compositions can be incorporated into particulate preparations of polymeric compounds such as polylactic acid, polyglycolic acid, etc., or into liposomes or micelles. Such compositions may influence the physical state, stability, rate of in vivo release, and rate of in vivo clearance of components of a pharmaceutical composition of the present invention. The pharmaceutical composition of the present invention can be prepared, for example, in liquid form, or can be in dried powder form (e.g., lyophilized). Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

The term “treat” as used herein refers to any type of treatment that imparts a benefit to a patient afflicted with a disease, including improvement in the condition of the patient (e.g., amelioration of one or more symptoms), delay in the progression of the condition, etc.

The following examples are provided to illustrate certain embodiments of the invention. They are not intended to limit the invention in any way.

### Example I

### Identification of a Predictive Gene Signature

### Material and Methods

### Patient Samples

Samples from bone marrow biopsies done at the time of enrollment were taken from patients. Mononuclear cells were isolated by ficoll gradient separation and stored in liquid nitrogen. Response data was available for all patient samples.

### Power Calculations

The RNA sequencing experiment included samples derived from responders and non-responders for which differentially expressed genes (DEGs) were identified. Prior Illumina NextSeq RNA sequence data indicated that the minimum average read counts among well-detected genes is 50, the maximum dispersion is 0.4, and the ratio of the geometric mean of normalization factors is 1. Assuming a total number of −10,000 well-detected genes per sample (based on a target read depth of 50 million) and a desired minimum fold change of 3, we were able to reject the null hypothesis that the population means of the two groups are equal with 81% power (exact test). The FDR associated with this test is 0.05.

### RNA Isolation and Sequencing (RNAseq)

Total RNA was purified from bone marrow samples using the Qiagen RNeasy kit. RNA integrity (RIN) was determined by electrophoretic tracing of 200 ng total RNA using an Agilent Bioanalyzer. RNAseq libraries were constructed for samples with RIN scores >6.0 using the Illumina TruSeq Stranded Total RNA kit with Ribo-Zero rRNA depletion (1.0 ug total RNA as input). Indexed libraries were sequenced using an Illumina NextSeq 500 DNA sequencer using 150×150 nt paired end reads. Pooling 8 libraries per flow cell, we generated >50 million reads per sample with >75% of sequences achieving >Q30 Phred quality score. This sequencing depth and quality was optimal for analysis of differentially expressed genes and allele specific gene expression (31), and the read lengths were sufficient to detect splice variations, gene fusions, point mutations, in/dels and lncRNAs.

### RNAseq Data Analysis

Alignment of reads was performed using the STAR sequence aligner (32), and gene counts determined using feature Counts software (33). Differential gene expression was analyzed using DESeq2 software (34). All procedural and experimental conditions were held constant across samples to minimize technical variance and maximize power to detect DEGs via DESeq2 as described (35). Significant DEGs were conservatively defined as p<0.05 after adjustment for false discovery (Benjamini-Hochberg). DEGs were analyzed for significant enrichment of biological pathways and signaling networks using Ingenuity Pathway Analysis (IPA), Causal Network Analysis and Upstream Regulator Tools (36) and the DAVID software as described (37). Furthermore, DEGs found to differ between responders and non-responders were evaluated for their potential to form accurate, multi-gene prognostic models for classifying responders and non-responders. Models were constructed using multivariate classification algorithms we have previously employed (38, 39) including support vector machines (SVM), statistically weighted syndromes (SWS) and diagonal linear discriminant analysis (DLDA). In each instance, doubly-nested, leave-one-out cross validation were performed to construct fixed statistical models for class prediction. Sensitivity, specificity, PPV and NPV were assessed on held out cases as an estimate of the classification power of the models we develop.

### Results

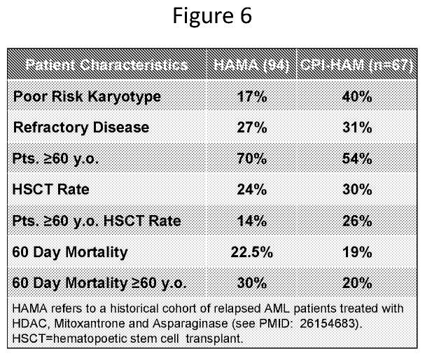

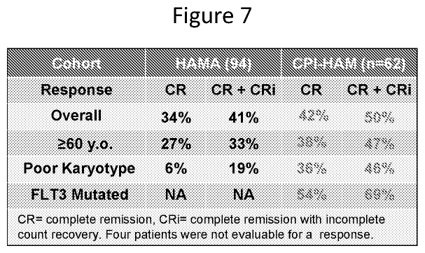

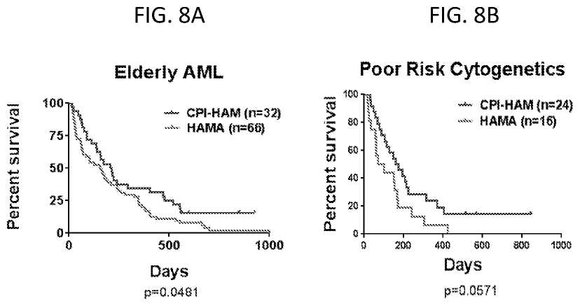

In a recently completed phase I study, CPI-613 was given in combination with high dose cytarabine and mitoxantrone to patients with relapsed or refractory AML (FIGS. 6-7). In this trial, 67 patients were enrolled and 62 were evaluable. Four patients did not undergo a post-treatment bone marrow biopsy and one patient with accelerated phase CML was enrolled in error. The median age was 60 years (range 21-79). In patients with relapsed disease the median duration of CR1 was 11.2 months. Cytogenetics were poor risk in 27 patients, intermediate in 33, and good in 6. In the 62 evaluable patients the results were as follows. The overall response rate was 50% (26CR+5CRi) with a median survival of 6.7 months. Response was significantly associated with survival with a median survival of 13.2 months in responders compared to 3 months for all others. When compared to historical controls treated with the same chemotherapy without CPI-613 an increase in response rate was seen. This was especially prominent in patients with poor risk disease where the response rate more than doubled (28). In the historical cohort of patients only 19% of poor risk karyotype patients had a response. Surprisingly, the response rate for patients with poor risk cytogenetics was 46% (9CR+2CRi/24) (FIG. 7). Additionally, the median survival of the historical patients was only 2.8 months while the median survival in the CPI-613 treated patients was 5.5 months (FIG. 8B). Median survival was also improved in an elderly cohort (FIG. 8A). In patients >60 years old the CR/CRi rate was 47% (15/32) with a median survival of 6.9 months (FIG. 8A). This was not significantly different from patients younger than 60 years old. While the reason for this dramatic improvement in response remains unknown, these data demonstrate that patients with AML that are more likely to respond to the combination of CPI-613 and chemotherapy can be identified on a genetic basis.

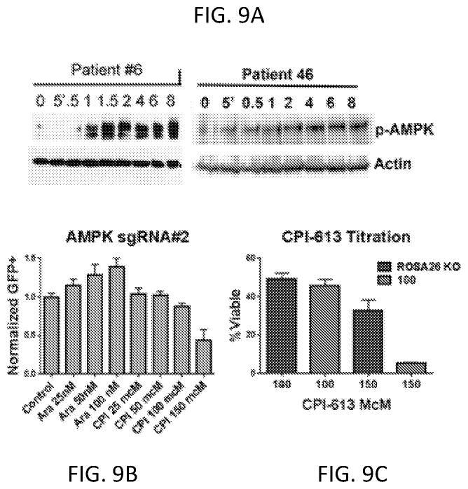

Several patients had blood samples taken before and after CPI-613 infusion. Three of these patients had increased phosphorylation of PDH consistent with its inhibition. Additionally, two patients demonstrated a robust phosphorylation of AMPK consistent with preclinical studies (FIG. 9A). The role of AMPK in response was assessed by competition assays using Cas-9 expressing AML cells that have deleted AMPK (FIG. 9B). Normal cells out-competed AMPK deleted cells when treated with CPI-613 but not when treated with cytarabine (FIG. 9C), suggesting AMPK is a specific resistance factor against TCA cycle inhibition.

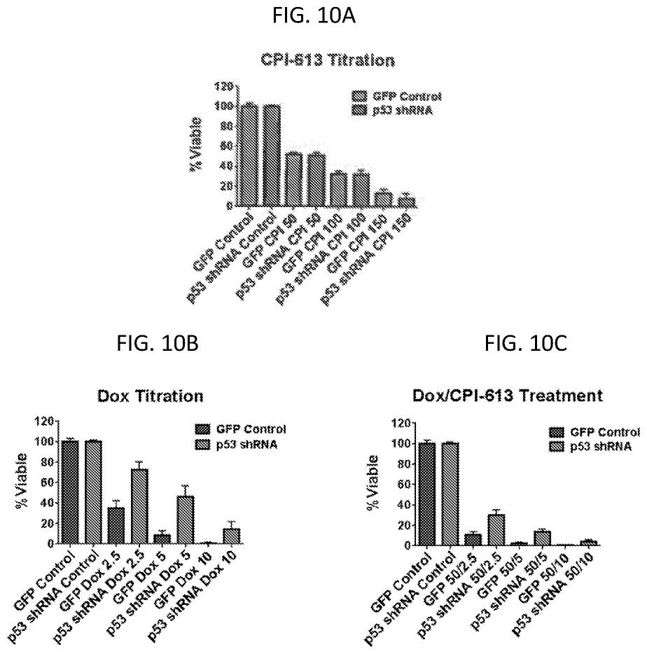

Loss of p53 is associated with poor risk cytogenetics, poor prognosis and resistance to chemotherapy but not resistance to CPI-613. In AML patients poor risk karyotypes are associated with p53 loss or mutation in 70-80% of cases (13, 29). Indeed it has been argued that much of the prognostic importance of poor risk karyotypes is mediated by p53 loss (13). This is not surprising given the association between p53 status and response to chemotherapy in AML (30). In order to assess the consequences of p53 suppression on mitochondrial inhibition we generated murine AML cells that knocked down p53 by shRNA. As predicted these cells became significantly resistant to doxorubicin compared to vector controls (FIG. 10B). Surprisingly, there was no change in response to CPI-613 (FIG. 10A) and the presence of CPI-613 increased sensitivity to doxorubicin to that of the controls (FIG. 10C). These findings suggest that mitochondrial inhibition can sensitize p53 suppressed AML cells to DNA damage and may be the reason for the more than doubling of the response rate seen in poor risk relapsed or refractory AML patients treated with CPI-613 in combination with chemotherapy.

Despite the fact that the combination of chemotherapy and CPI-613 has impressive activity compared to historical controls, not all patients respond. The ability to prospectively identify patients with the highest probability of a response allows for streamlined and efficient clinical development, spares patients unlikely to benefit from the therapy, and provides mechanistic insights not otherwise attainable. Accordingly, we have identified a gene expression signature that predicts response to treatment modalities that include CPI-613, particularly treatment protocols combining high dose cytarabine, mitoxantrone and CPI-613.

To gain insight into the transcriptional programs that distinguish responders from non-responders prior to treatment, we sequenced the transcriptomes of baseline bone marrow mononuclear cells obtained from a selection of responding and non-responding relapsed or refractory acute myeloid leukemia treated with the combination of high dose cytarabine (HDAC), mitoxantrone, and CPI-613. As a result, an RNAseq signature was derived from 25 baseline patient samples: 19 from bone marrow and 9 from peripheral blood. These samples corresponded to 14 responders and 11 non-responders. We focused our analysis on the 19 bone marrow samples (since bone marrow represents the most clinically rigorous biopsy site for leukemia). Quality control analysis of the sequencing data identified two cases with insufficient sequence read quality correlating with high degradation and low yield of RNA. Thus, 17 samples (10 responders and 7 non-responders) were deemed reliable for downstream biomarker analysis.

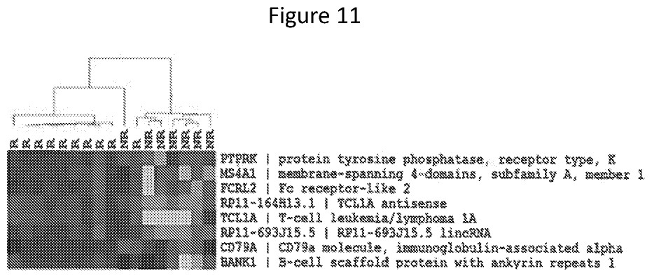

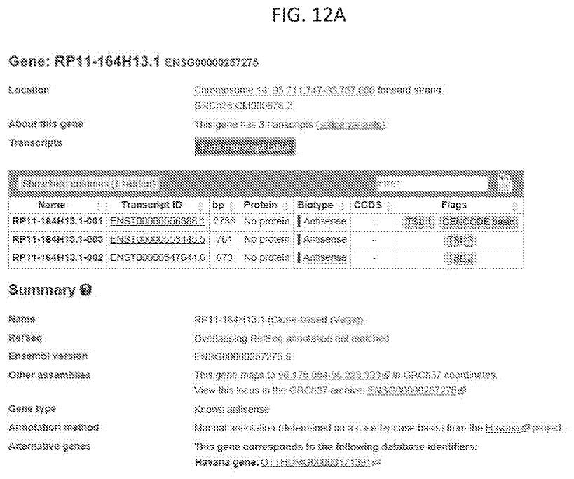

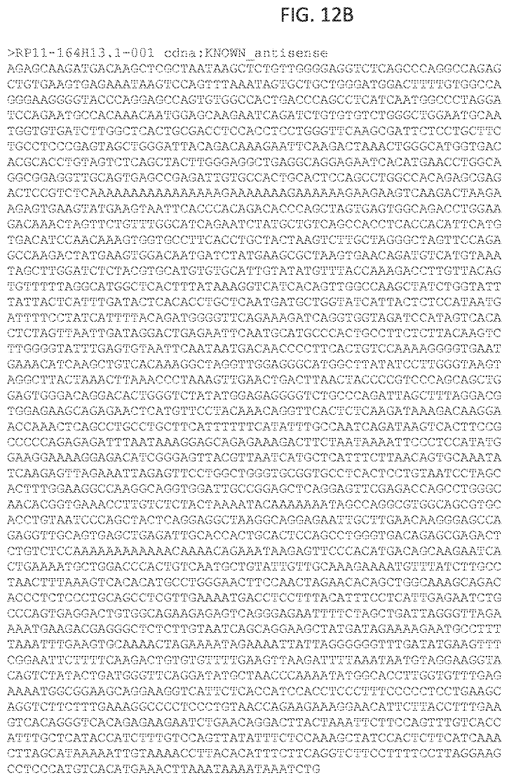

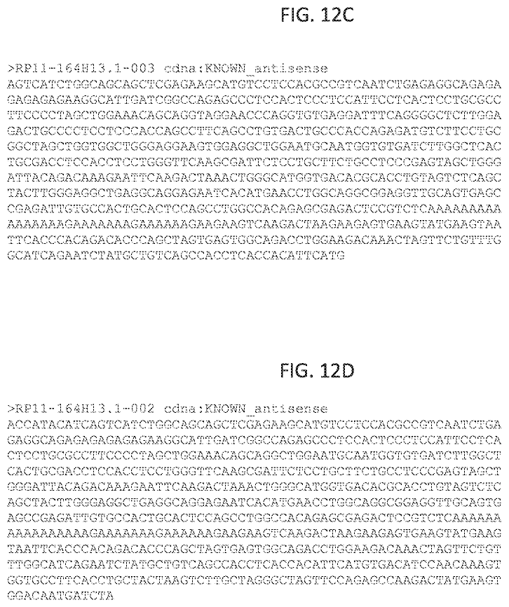

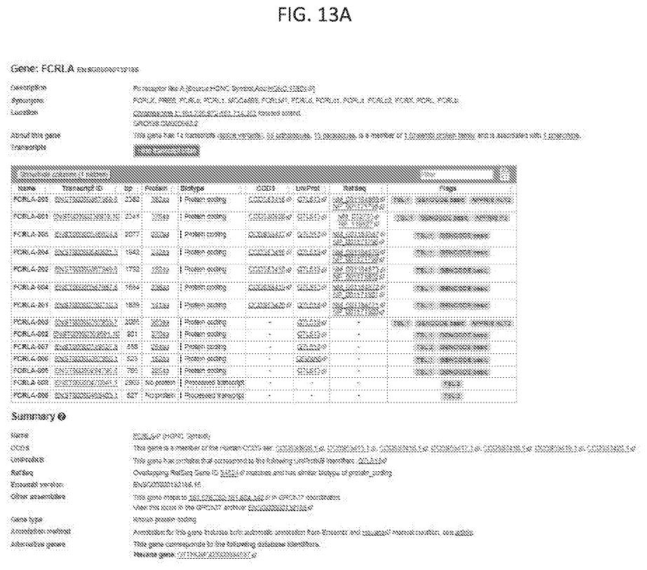

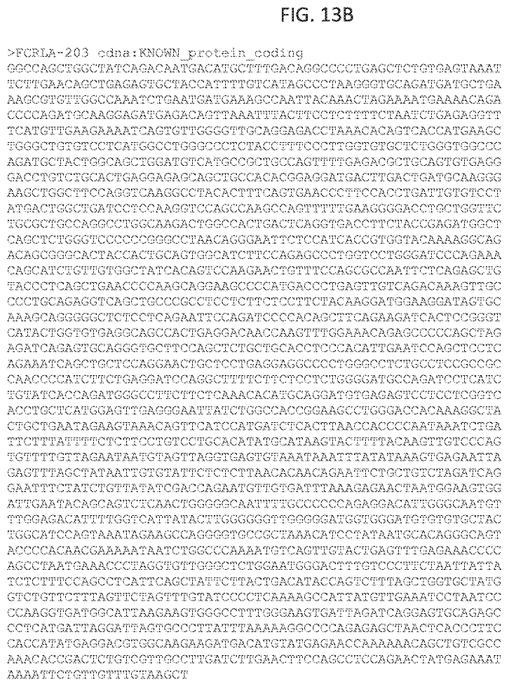

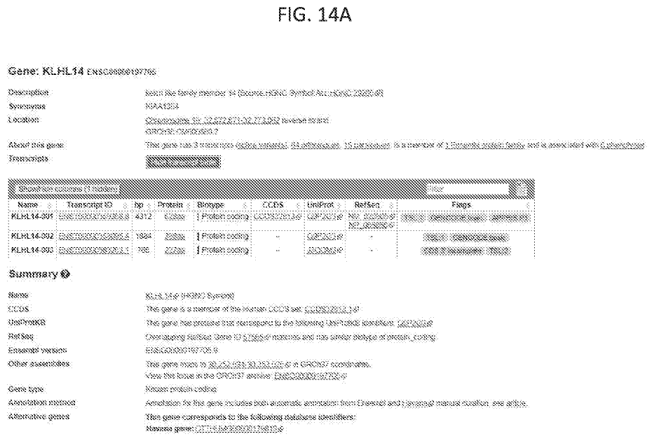

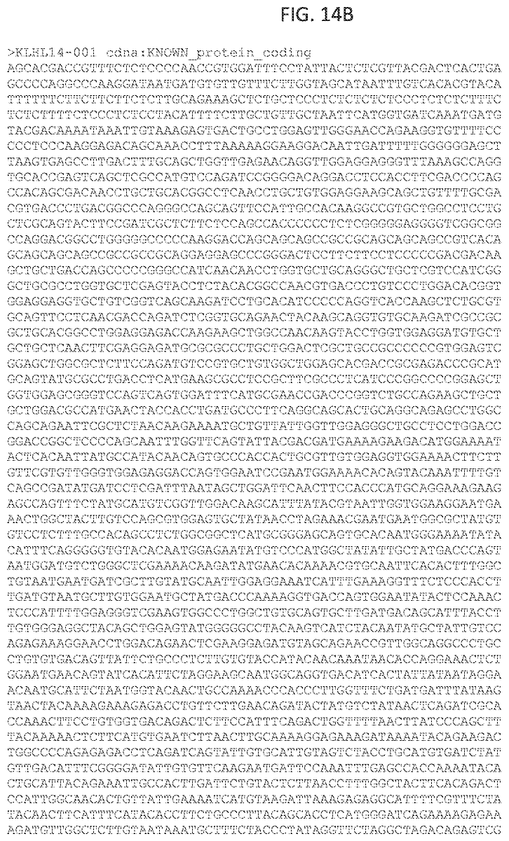

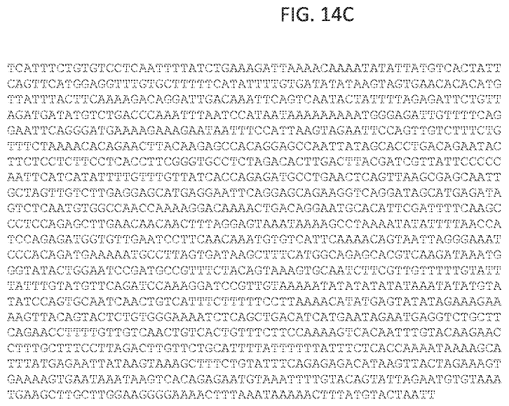

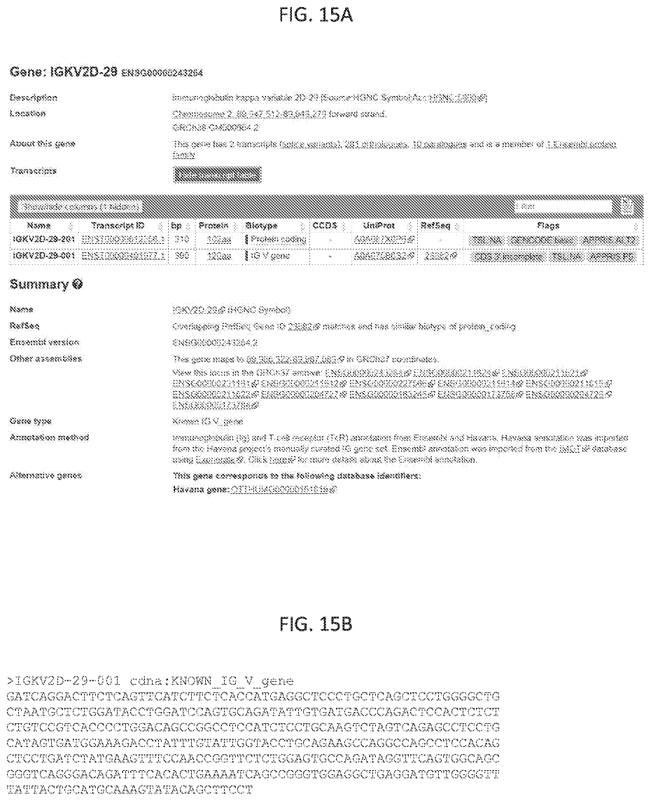

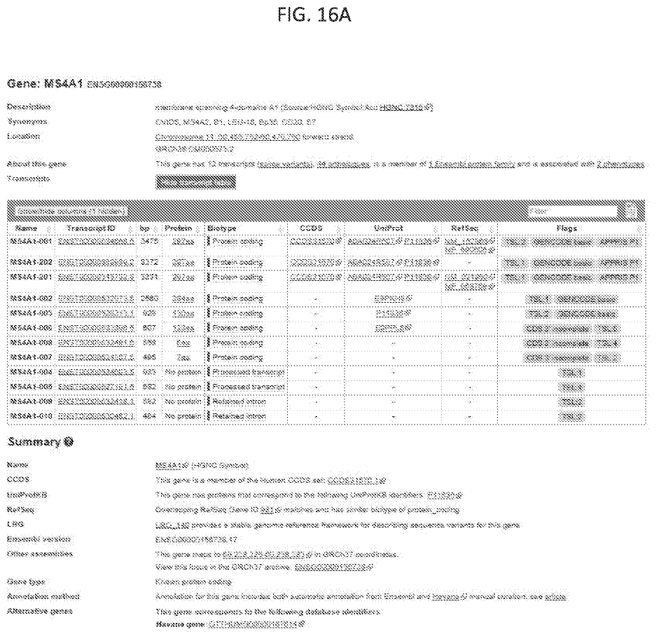

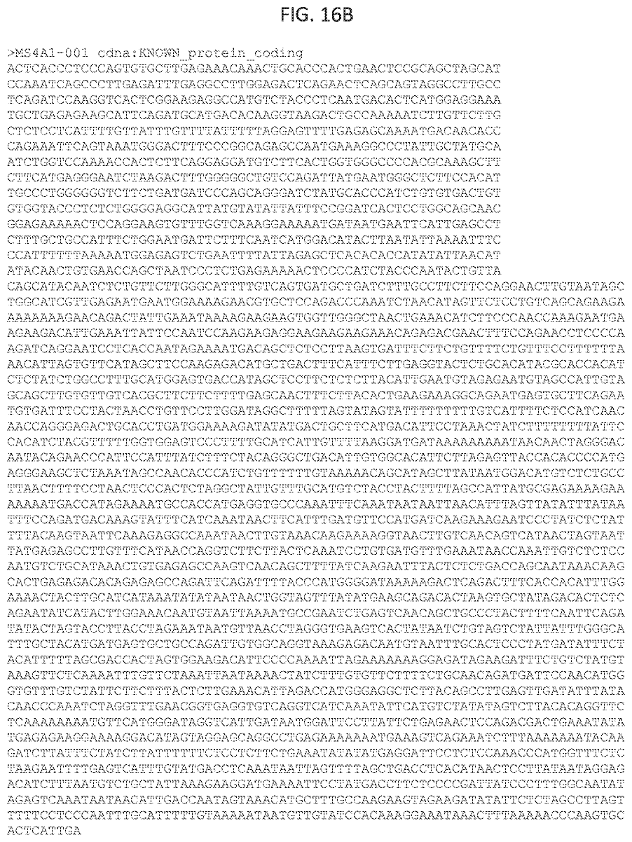

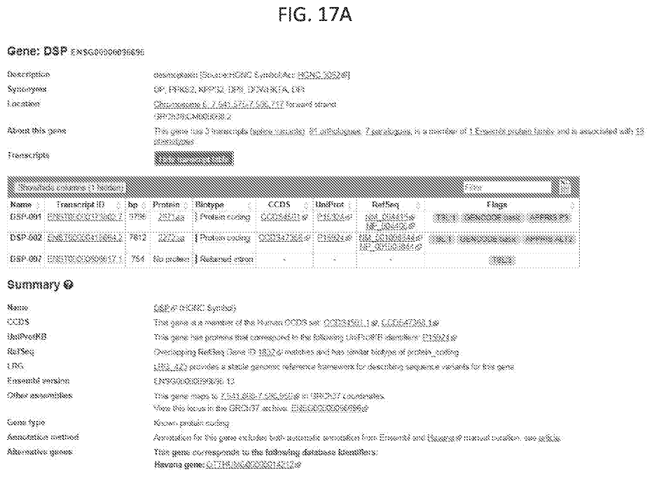

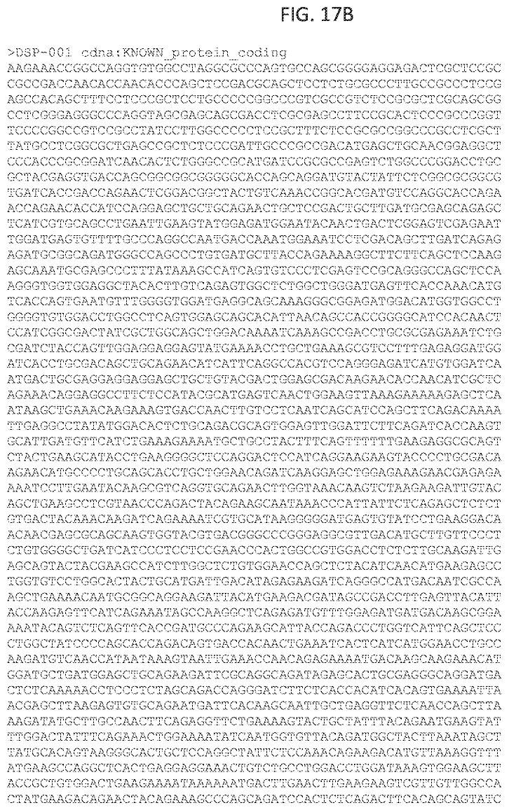

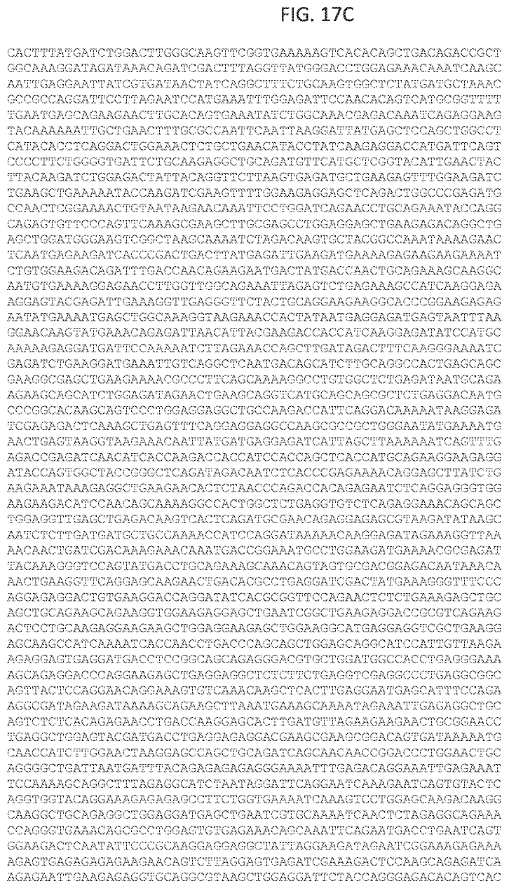

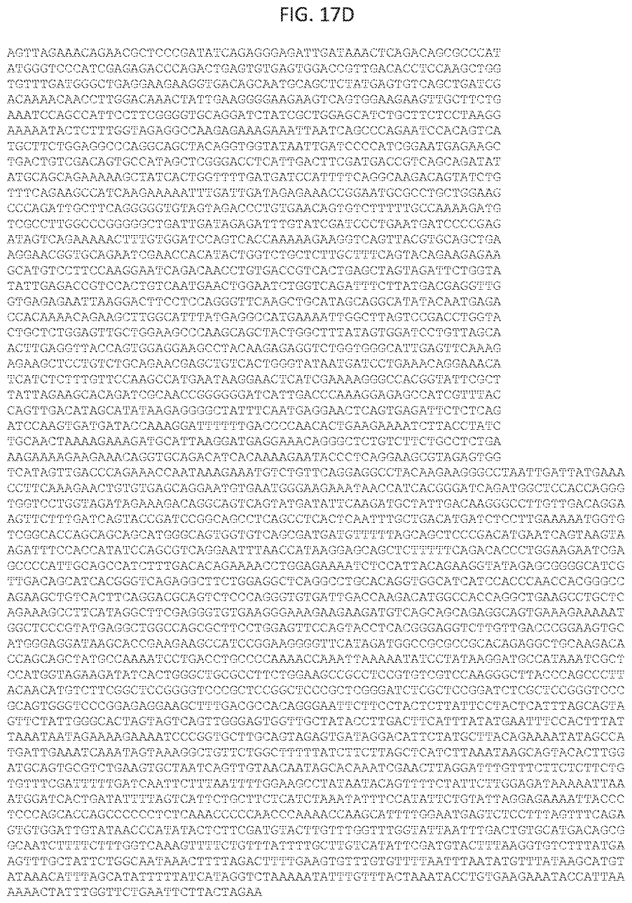

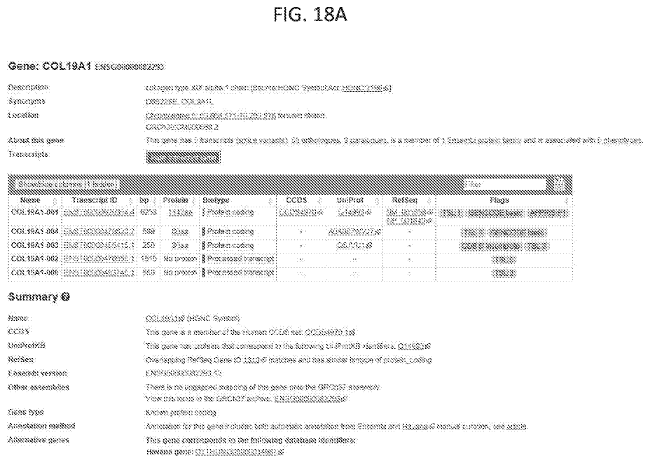

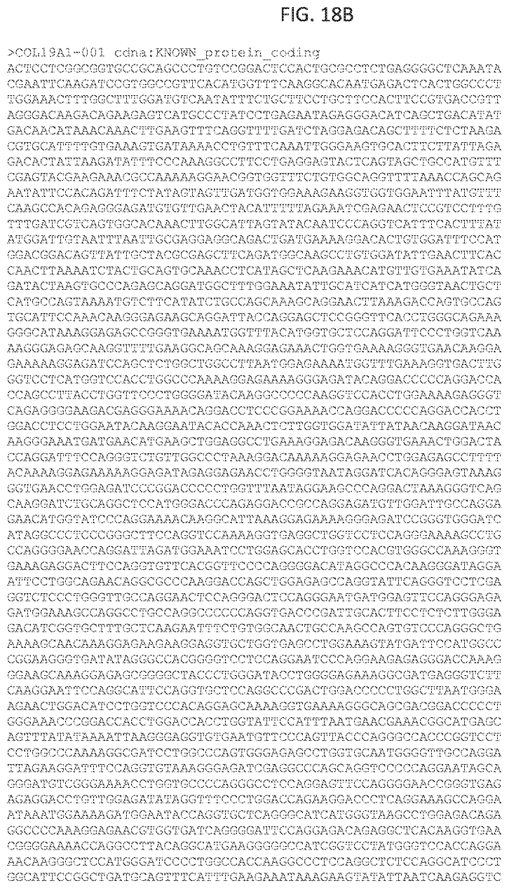

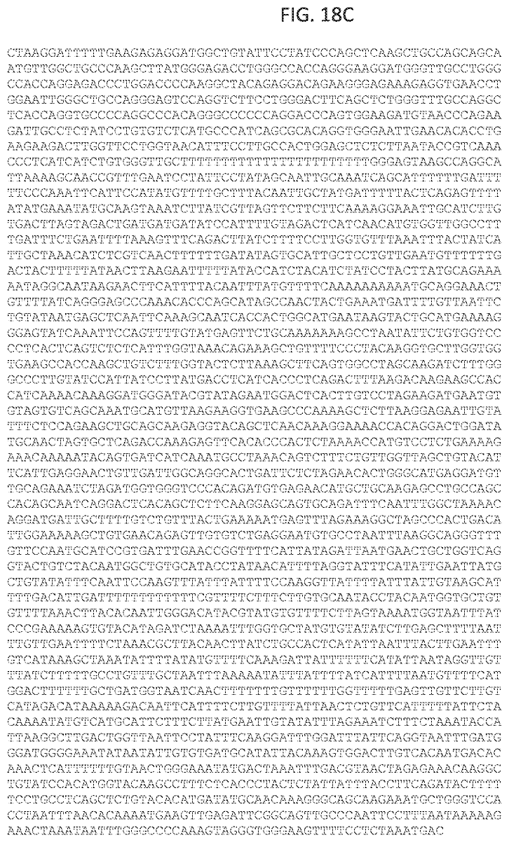

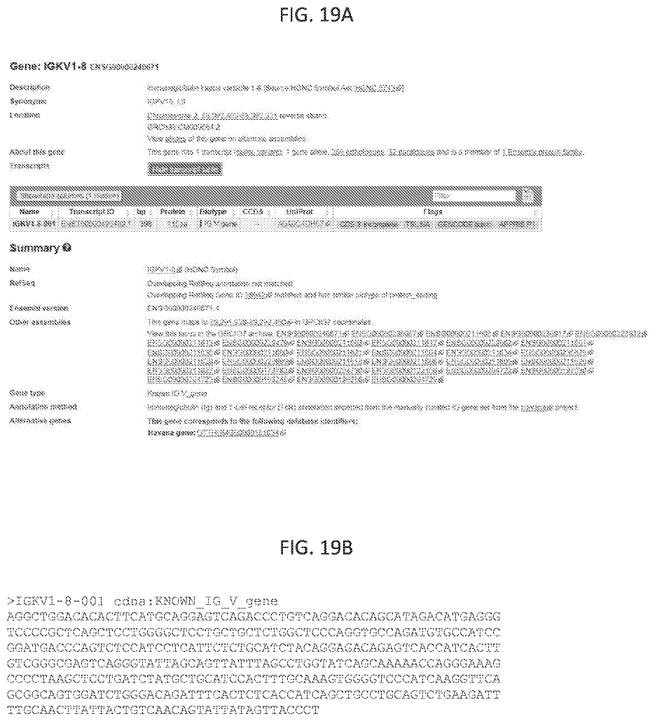

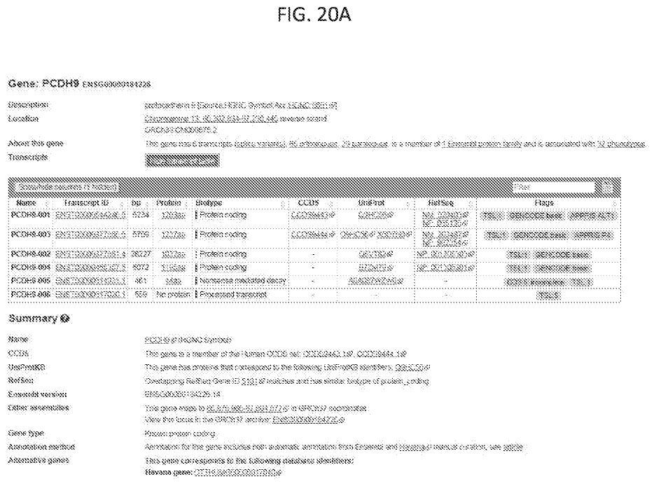

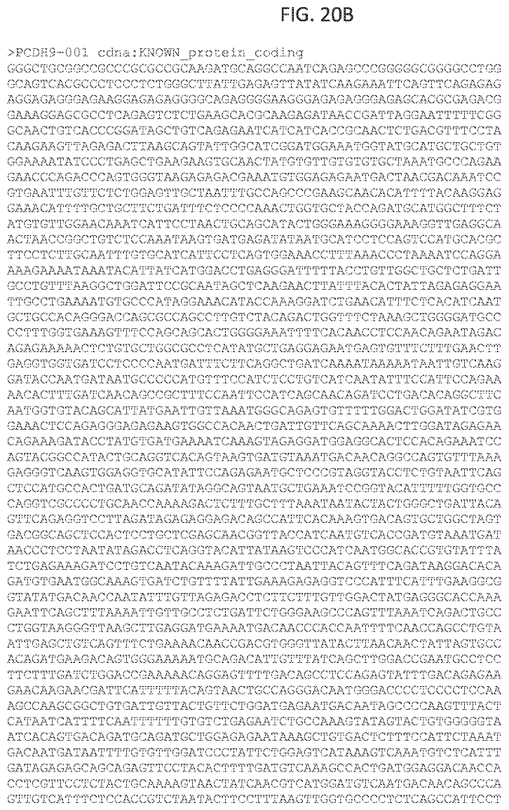

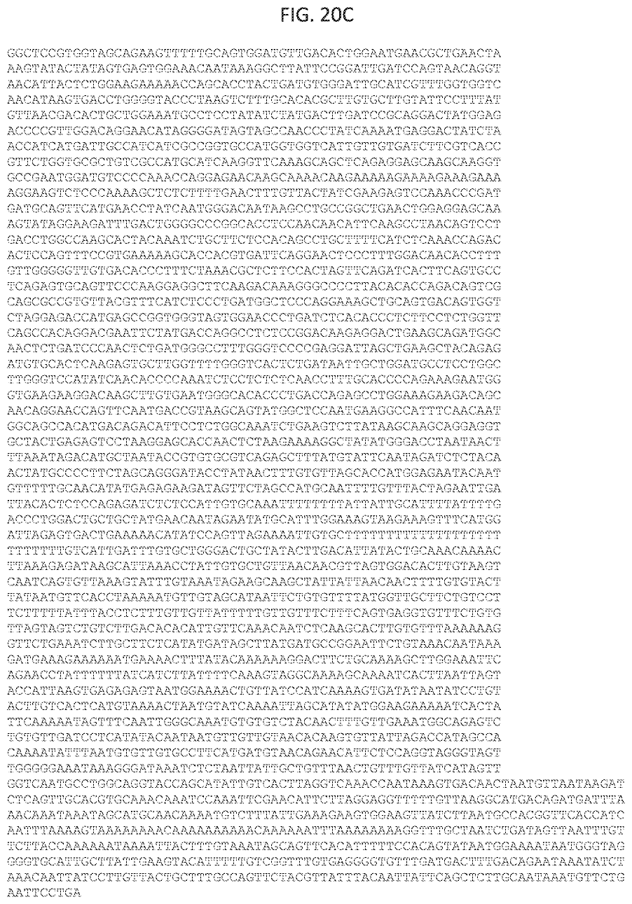

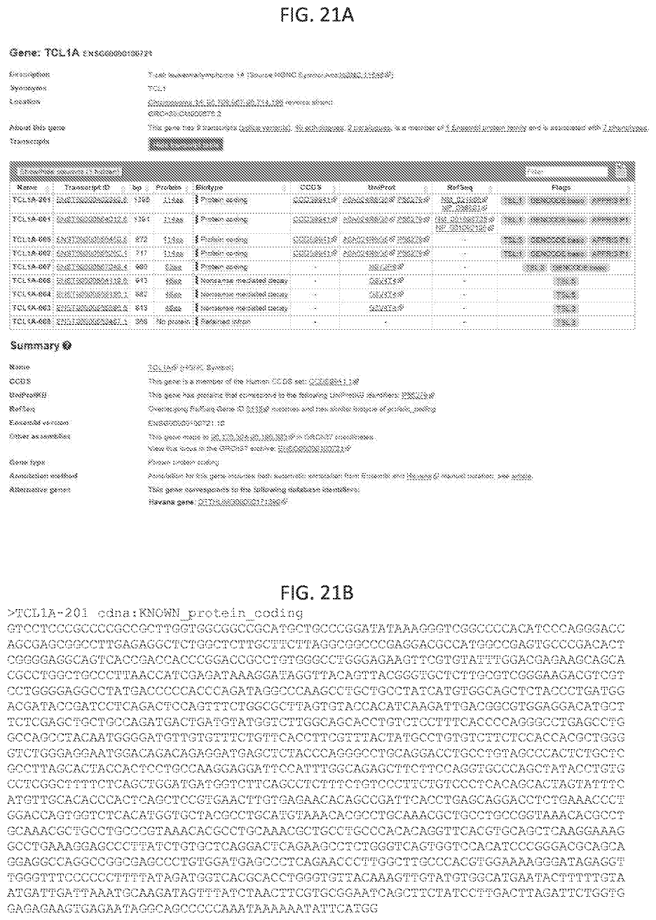

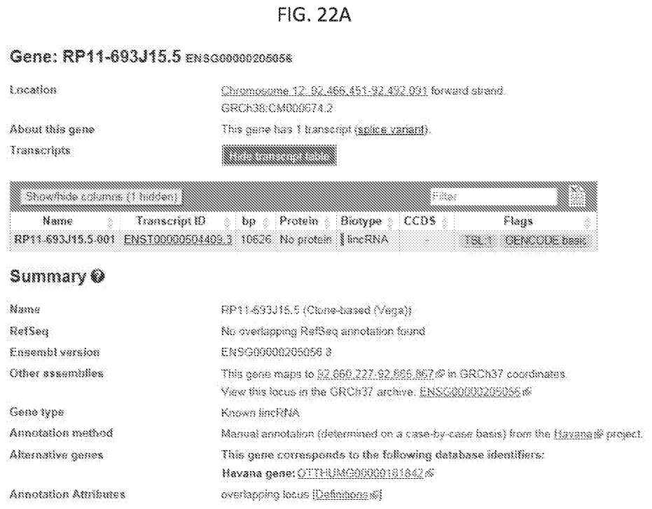

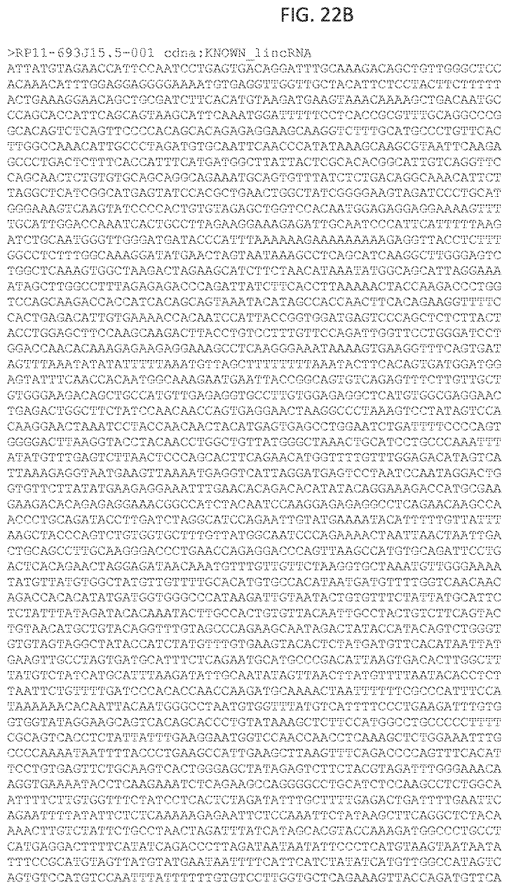

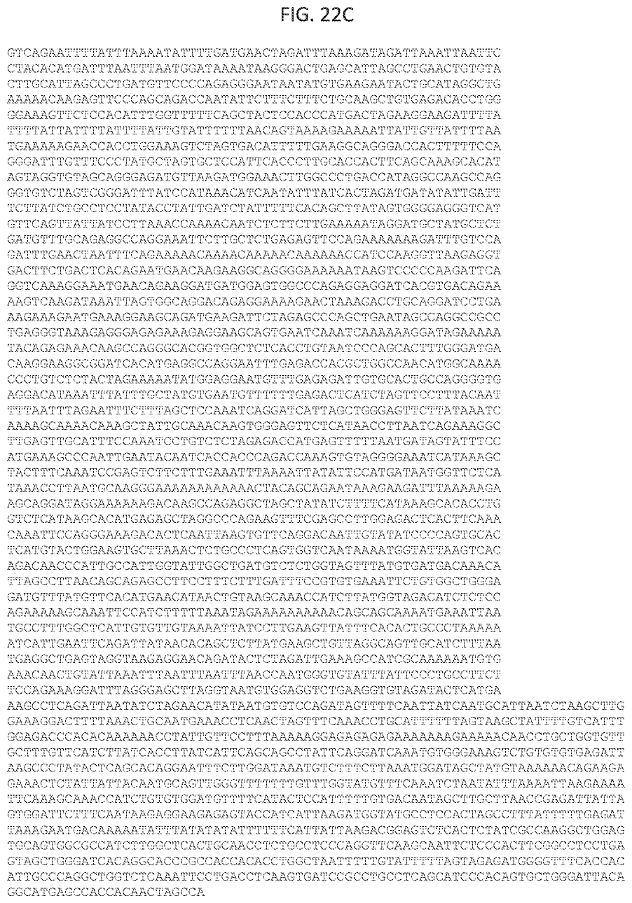

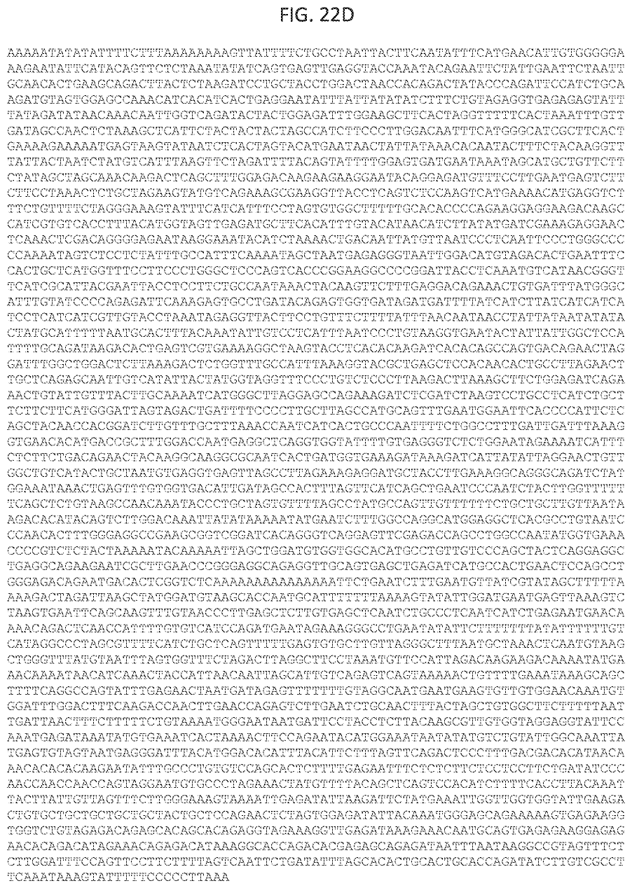

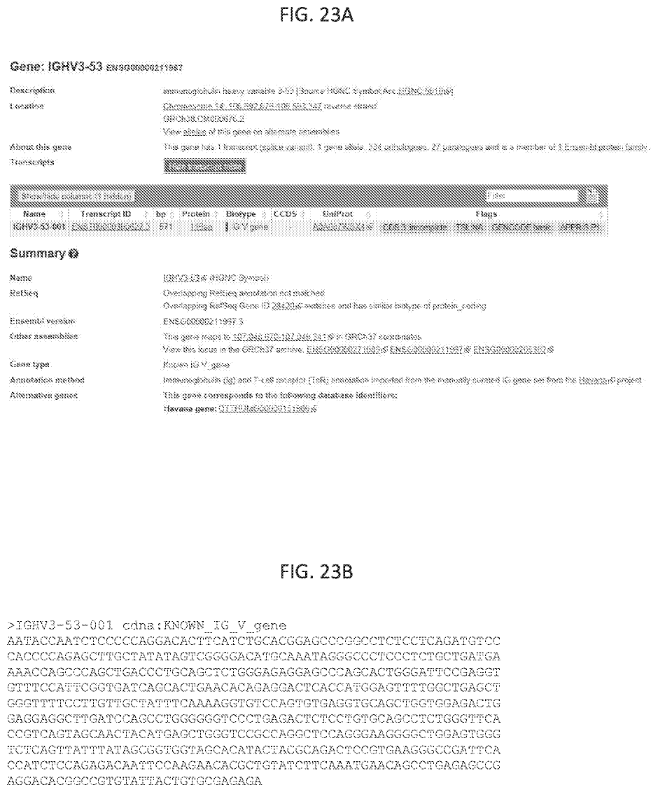

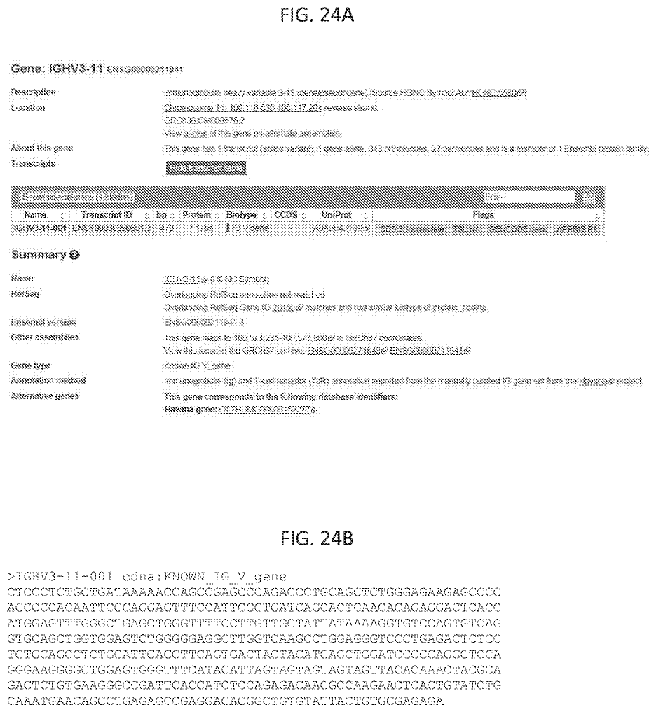

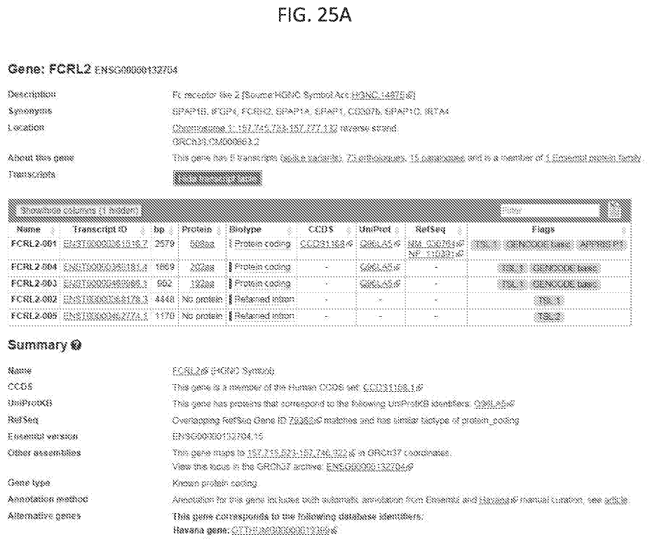

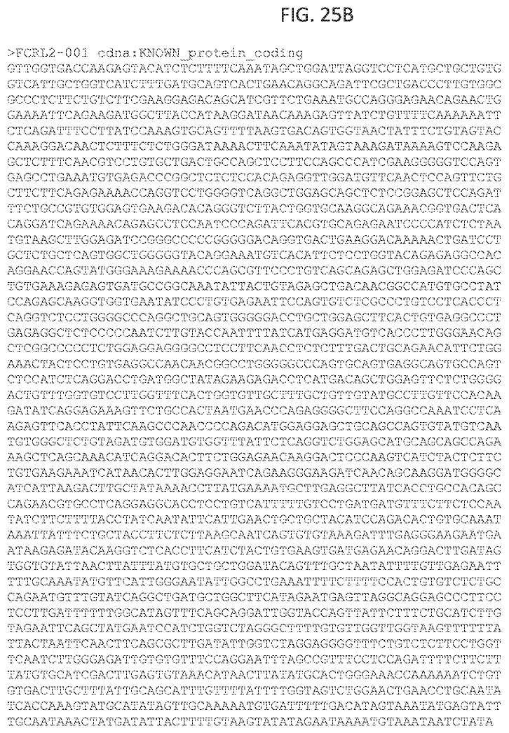

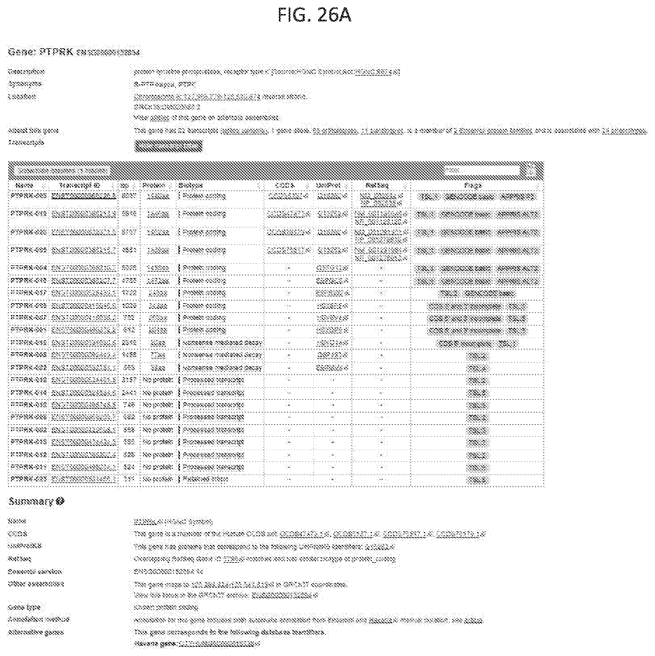

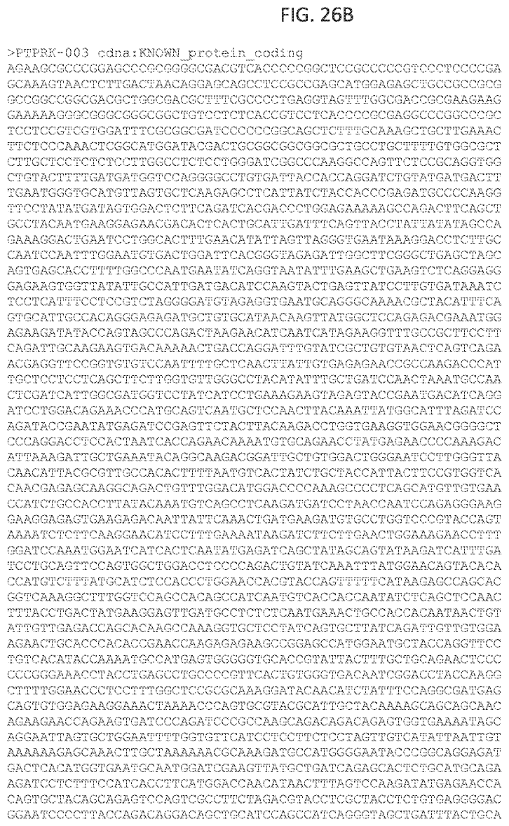

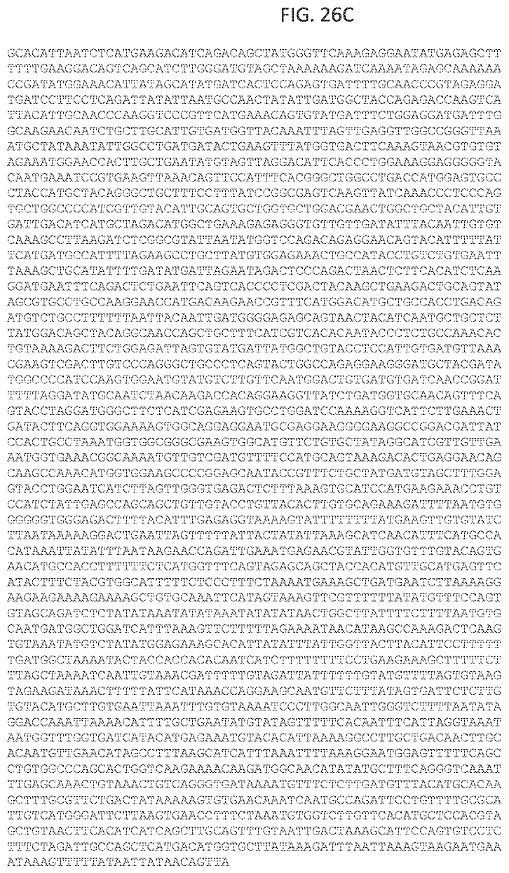

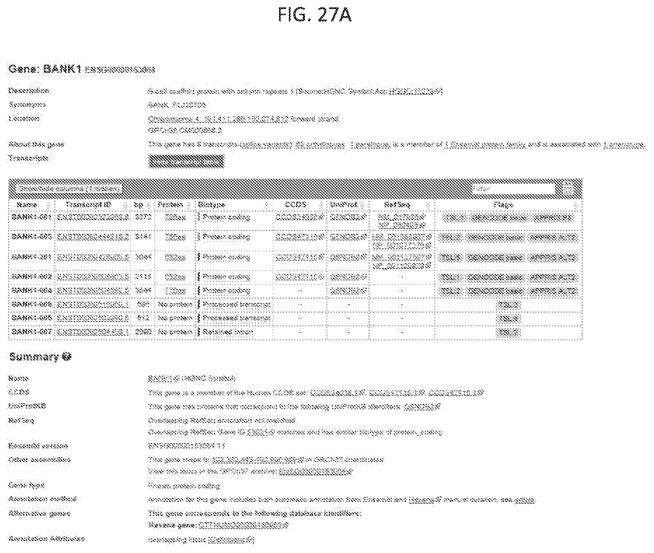

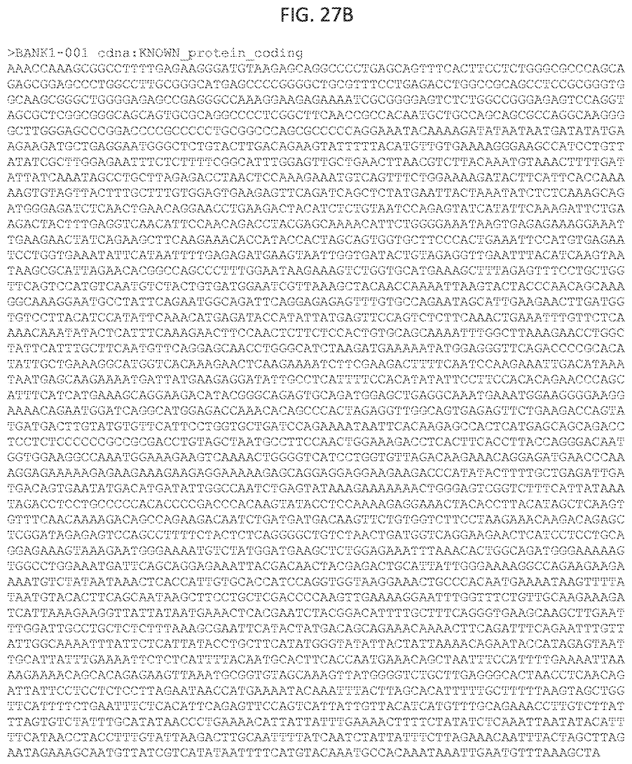

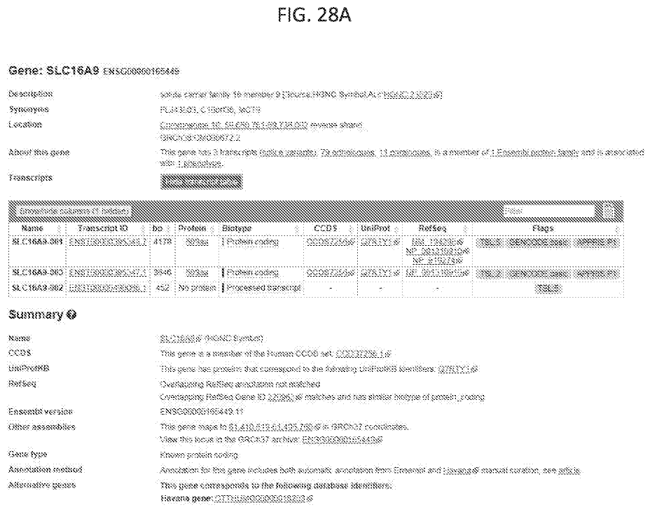

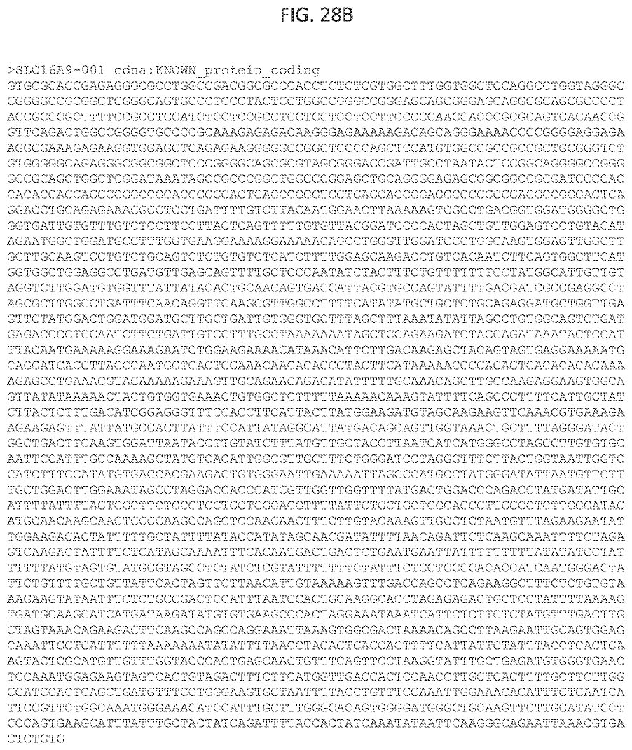

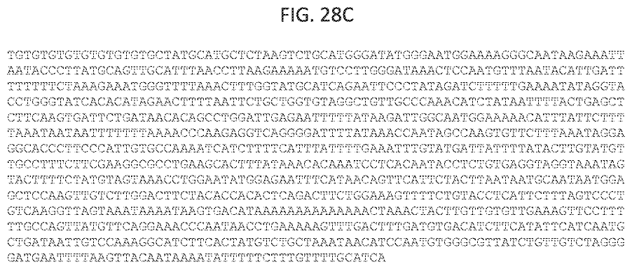

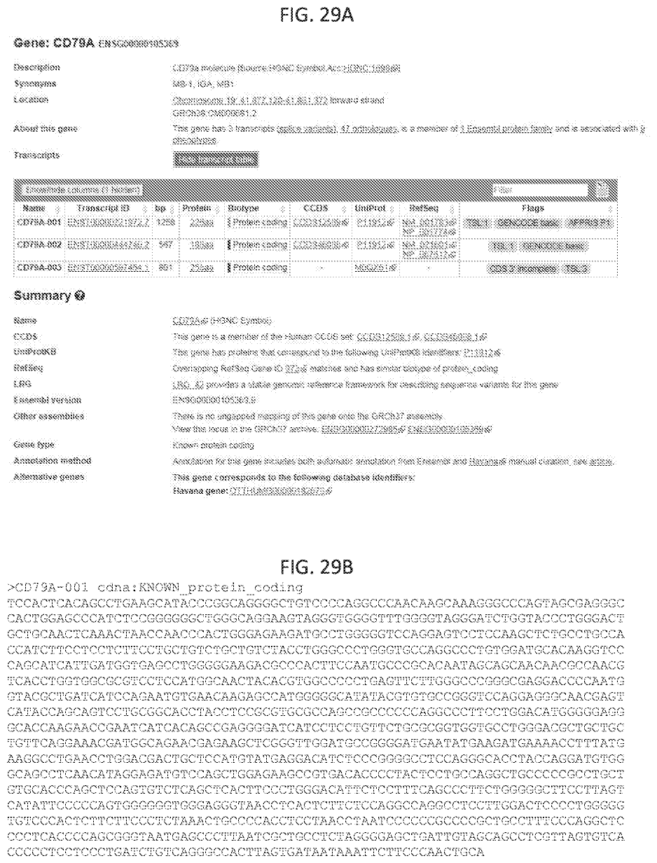

Analysis of patient samples led to the identification of predictive genes with significantly increased expression in responding patients (Table 1 and FIGS. 12-29). Analysis for enrichment of gene ontologies revealed several significant biological categories for the genes overexpressed in responders, while none were identified for the non-responders. The responders showed significant enrichment for gene categories related to immune involvement, particularly genes associated with B lymphocytes. Genes comprising the B cell signature include TCL1A, MS4A1, FCRL2, PTPRK, CD79A, BANK1, RP11-164H13.1 (TCL1A antisense) and RP11-693J15.5 (lincRNA). Hierarchical cluster analysis of these genes segregates patients into two groups, those with low expression of the signature genes, and those with high expression of the signature genes (FIG. 11). Only two samples, 1 responder (R) and 1 non-responder (NR) misclassified owing to gene expression levels in between that of the two groups, suggesting a sensitive and specific threshold for class prediction.

TABLE 1

| Ensembl ID | Gene | Gene Description | log2FoldChange |
|---|---|---|---|
| ENSG00000257275 | RP11-164H13.1 | (TCL1A antisense) | 4.21 |
| ENSG00000132185 | FCRLA | Fc receptor-like A | 3.44 |
| ENSG00000197705 | KLHL14 | kelch-like family member 14 | 3.42 |
| ENSG00000243264 | IGKV2D-29 | immunoglobulin kappa variable 2D-29 | 3.31 |
| ENSG00000156738 | MS4A1 | membrane-spanning 4-domains, | 3.02 |
| | | subfamily A, member 1 | |
| ENSG00000096696 | DSP | desmoplakin | 2.96 |
| ENSG00000082293 | COL19A1 | collagen, type XIX, alpha 1 | 2.93 |
| ENSG00000240671 | IGKV1-8 | immunoglobulin kappa variable 1-8 | 2.90 |
| ENSG00000184226 | PCDH9 | protocadherin 9 | 2.80 |
| ENSG00000100721 | TCL1A | T-cell leukemia/lymphoma 1A | 2.76 |
| ENSG00000205056 | RP11-693J15.5 | (lincRNA) | 2.70 |
| ENSG00000211967 | IGHV3-53 | immunoglobulin heavy variable 3-53 | 2.66 |
| ENSG00000211941 | IGHV3-11 | immunoglobulin heavy variable 3-11 | 2.56 |
| | | (gene/pseudogene) | |
| ENSG00000132704 | FCRL2 | Fc receptor-like 2 | 2.52 |
| EN5G00000152894 | PTPRK | protein tyrosine phosphatase, receptor | 2.23 |
| | | type, K | |
| ENSG00000153064 | BANK1 | B cell scaffold protein with ankyrin | 2.14 |
| | | repeats 1 | |
| ENSG00000165449 | SLC16A9 | solute carrier family 16, member 9 | 2.12 |
| ENSG00000105369 | CD79A | CD79a molecule, immunoglobulin- | 1.92 |
| | | associated alpha | |

### Example II

### Detection of B Cell Infiltration

Development of a predictive gene expression signature from responding and non-responding patients has led to the identification of approximately 18 genes with significantly increased expression levels in responding patients (Table 1 and FIGS. 12-29). Pathway enrichment analysis revealed a significant enrichment for genes involved in immune processes related to mature B cell and plasma B cell biology, including the canonical B cell marker genes, CD20 (MS4A1) and CD79A. Various approaches can be used to translate these findings into a diagnostic test for predicting treatment responses.

One method relies on detection of gene products as an indication of the relative abundance of infiltrating B cell populations in a patient sample. This approach entails immunohistochemical (IHC) staining or flow cytometric analysis of clinically obtained bone marrow or peripheral blood samples for detection of genes or markers identified, in particular B cell lineage markers. For example, CD20 protein expression can be used quantify the numbers of CD20-positive cells in a patient sample and predict patient response.

Another approach involves diagnostic RNA profiling on the NanoString n-Counter platform. The nCounter platform employs a digital, color-coded barcode technology that allows the direct counting of hundreds of unique barcoded gene transcripts in a single solution-based reaction. The method enables high precision and sensitivity. Code sets (barcoded oligonucleotides that “capture” the transcripts of interest) can be designed against our genes of interest, incubated with bone marrow or peripheral blood-derived cell lysates (with or without prior RNA isolation) or, alternatively, isolated RNA of mass 100-400 nanograms, and processed for digital quantitation of transcripts.

### Example III

### Test and Treat Method for Ameliorating Symptoms Associated with AML

In order to treat an individual having AML, to alleviate a sign or symptom of the disease, CPI-613 and suitable chemotherapeutic agents can be administered in combination in order to provide therapeutic benefit to the patient. Such agents are administered in an effective dose.

First, a biological sample is obtained from a patient. Nucleic acids present in the sample are then assessed for the expression levels of one or more genes indicated in Table 1. The presence of elevated levels of these genes indicates sensitivity to CPI-613 treatment and provides the clinician with guidance as to which therapeutic agents are appropriate. The total treatment dose or doses can be administered to a subject as a single dose or can be administered using a fractionated treatment protocol, in which multiple/separate doses are administered over a more prolonged period of time, for example, over the period of a day to allow administration of a daily dosage or over a longer period of time to administer a dose over a desired period of time. One skilled in the art would know that the amount of CPI-613 and chemotherapeutic agents required to obtain an effective dose in a subject depends on many factors, including the age, weight and general health of the subject, as well as the route of administration and the number of treatments to be administered. The dosages described herein are generally those for an average adult but can be adjusted for the treatment of children. The dose will generally range from about 0.001 mg to about 1000 mg. In view of these factors, the skilled artisan would adjust the particular dose so as to obtain an effective dose for treating an individual having AML.

In an individual suffering from AML, in particular a more severe form of the disease, administration of CPI-613 can be particularly useful when administered in combination, for example, with a conventional agent for treating such a disease. The skilled artisan would administer CPI-613 alone or in combination and would monitor the effectiveness of such treatment using routine methods, such as pulmonary, inflammatory function determination, radiologic, immunological assays, or histopathological methods.

Administration of the pharmaceutical preparation is preferably in an “effective amount,” this being an amount sufficient to show benefit to the individual. This amount prevents, alleviates, abates, or otherwise reduces the severity of AML symptoms in a patient.

In a preferred embodiment of this invention, a method is provided for the synergistic treatment of CPI-613 using the chemotherapeutic agents disclosed in the present examples and those known by the skilled artisan in combinatorial approaches. Advantageously, the synergistic method of this invention reduces the development of AML, or reduces symptoms of AML in a mammalian host. Additionally, therapeutic regimens suitable for simultaneous treatment of two or more hematological disorders are also provided. Moreover, it is known that certain patients present with higher risk AML (e.g., poor risk cytogenetics). The information provided herein guides the clinician in new treatment modalities for the management of AML.

Methods for the safe and effective administration of most chemotherapeutic agents are known to those skilled in the art. In addition, their administration is described in the standard literature. For example, the administration of many of the chemotherapeutic agents is described in the “Physicians' Desk Reference” (PDR), e.g., 1996 edition (Medical Economics Company, Montvale, N.J. 07645-1742, USA); the disclosure of which is incorporated herein by reference thereto.

The present invention also encompasses a pharmaceutical composition useful in the treatment of AML, comprising the administration of a therapeutically effective amount of the combinations of this invention, with or without pharmaceutically acceptable carriers or diluents. The synergistic pharmaceutical compositions of this invention comprise two or more of the agents and a pharmaceutically acceptable carrier. The compositions of the present invention may further comprise one or more pharmaceutically acceptable additional ingredient(s) such as alum, stabilizers, antimicrobial agents, buffers, coloring agents, flavoring agents, adjuvants, and the like. The AML treatment compositions of the present invention may be administered orally or parenterally including the intravenous, intramuscular, intraperitoneal, subcutaneous, rectal and topical routes of administration.

Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired. Intermittent therapy (e.g., one week out of three weeks or three out of four weeks) may also be used.

Certain AML subjects can be treated effectively with a plurality of compounds. Such triple and quadruple combinations can provide greater efficacy. When used in such triple and quadruple combinations the dosages can be determined according to known protocols.

CPI-613 and its combinations with other chemotherapeutics agents may also be co-administered with other well known therapeutic agents that are selected for their particular usefulness against the condition that is being treated. Combinations of the instant invention may alternatively be used sequentially with known pharmaceutically acceptable agent(s) when a multiple combination formulation is inappropriate.

Also, in general, the compounds intended to treat AML do not have to be administered in the same pharmaceutical composition, and may, because of different physical and chemical characteristics, have to be administered by different routes. For example, first compound may be administered orally to generate and maintain good blood levels thereof, while a second compound may be administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

### REFERENCES

1. Licht J D, Sternberg O W. The molecular pathology of acute myeloid leukemia. Hematology Am Soc Hematol Educ Program. 2005:137-42. Epub 2005/11/24. doi: 2005/1/137 [pii] 10.1182/asheducation-2005.1.137. PubMed PMID: 16304371.
2. Dohner H, Estey E H, Amadori S, Appelbaum F R, Buchner T, Burnett A K, Dombret H, Fenaux P, Grimwade D, Larson R A, Lo-Coco F, Naoe T, Niederwieser D, Ossenkoppele G J, Sanz M A, Sierra J, Tallman M S, Lowenberg B, Bloomfield C D, European L. Diagnosis and management of acute myeloid leukemia in adults: recommendations from an international expert panel, on behalf of the European LeukemiaNet. Blood. 2010; 115(3):453-74. doi: 10.1182/blood-2009-07-235358. PubMed PMID: 19880497.
3. Estey E H. Acute myeloid leukemia: 2013 update on risk-stratification and management. American journal of hematology. 2013; 88(4):318-27. doi: 10.1002/ajh.23404. PubMed PMID: 23526416.
4. Yates J W, Wallace H J, Jr., Ellison R R, Holland J F. Cytosine arabinoside (NSC-63878) and daunorubicin (NSC-83142) therapy in acute nonlymphocytic leukemia. Cancer chemotherapy reports Part 1. 1973; 57(4):485-8. PubMed PMID: 4586956.
5. Tallman M S, Gilliland D G, Rowe J M. Drug therapy for acute myeloid leukemia. Blood. 2005; 106(4):1154-63. doi: 10.1182/blood-2005-01-0178. PubMed PMID: 15870183.
6. Bennett J M, Young M L, Andersen J W, Cassileth P A, Tallman M S, Paietta E, Wiernik P H, Rowe J M. Long-term survival in acute myeloid leukemia: the Eastern Cooperative Oncology Group experience. Cancer. 1997; 80(11 Suppl):2205-9. PubMed PMID: 9395035.
7. Keating M J, Kantarjian H, Smith T L, Estey E, Walters R, Andersson B, Beran M, Mccredie K B, Freireich E J. Response to salvage therapy and survival after relapse in acute myelogenous leukemia. Journal of clinical oncology: official journal of the American Society of Clinical Oncology. 1989; 7(8):1071-80. PubMed PMID: 2666590.
8. Ahmed T, Holwerda S, Klepin H D, Isom S, Ellis L R, Lyerly S, Manuel M, Dralle S, Berenzon D, Powell B L, Pardee T S. High dose cytarabine, mitoxantrone and 1-asparaginase (HAMA) salvage for relapsed or refractory acute myeloid leukemia {AML) in the elderly. Leukemia resear ch. 2015; 39(9):945-9. doi: 10.1016/j.leukres.2015.05.010. PubMed PMID: 26154683; PMCJD: PMC4546894.
9. Ravandi F. Relapsed acute myeloid leukemia: Why is there no standard of care? Best Pract Res Cl Ha. 2013; 26(3):253-9. doi: DOI 10.1016/j.beha.2013.10.005. PubMed PMID: WOS: 000329381000004.
10. Rowe J M, Li X C, Cassileth P A, Appelbaum F R, Schiffer C A, Wiernik P H, Litzow M R, Cripe L O, Lazarus H M, Paietta E, Dewald G W, Weinstein H J, Ogden A K, Woods W G, Shepherd L, Feusner J H, Bloomfield C D, Tallman M S. Very poor survival of patients with AML who relapse after achieving a first complete remission: The eastern cooperative oncology group experience. Blood. 2005; 106(11):162a-3a. PubMed PMID: WOS:000233426001015.
11. Roboz G J, Rosenblat T, Arellano M, Gobbi M, Altman J K, Montesinos P, O'Connell C, Solomon S R, Pigneux A, Vey N, Hills R, Jacobsen T F, Gianella-Borradori A, Foss O, Vetrhusand S, Giles F J. International randomized phase Ill study of elacytarabine versus investigator choice in patients with relapsed/refractory acute myeloid leukemia. Journal of clinical oncology: official journal of the American Society of Clinical Oncology. 2014; 32 (18):1919-26. doi: 10.1200/J C0.2013.52.8562. PubMed PMID: 24841975.
12. Byrd J C, Mrozek K, Dodge R K, Carroll A J, Edwards C G, Arthur D C, Pettenati M J, Patil S R, Rao K W, Watson M S, Koduru P R, Moore J O, Stone R M, Mayer R J, Feldman E J, Davey F R, Schiffer C A, Larson R A, Bloomfield C D, Cancer, Leukemia Group B. Pretreatment cytogenetic abnormalities are predictive of induction success, cumulative incidence of relapse, and overall survival in adult patients with de novo acute myeloid leukemia: results from Cancer and Leukemia Group B (CALGB 8461). Blood. 2002; 100(13)4325-36. Doi: 10.1182/blood-2002-03-0772. PubMed PMID: 12393746.
13. Rucker F G, Schlenk R F, Bullinger L, Kayser S, Teleanu V, Kett H, Habdank M, Kugler C M, Holzmann K, Gaidzik V I, Paschka P, Held G, van Lilienfeld-Toal M, Lubbert M, Frohling S, Zenz T, Krauter J, Schlegelberger B, Ganser A, Lichter P, Dohner K, Dehner H. T P53 alterations in acute myeloid leukemia with complex karyotype correlate with specific copy number alterations, monosomal karyotype, and dismal outcome. Blood. 2012; 119(9):2114-21. doi: 10.1182/blood-2011-08-375758. PubMed PMID: 22186996.
14. Jones R G, Thompson C B. Tumor suppressors and cell metabolism: a recipe for cancer growth. Genes Dev. 2009; 23(5):537-48. Epub 2009/03/10. doi: 23/5/537 [pii] 10.1101/gad.1756509. PubMed PMID: 19270154; PMCID: 2763495.
15. Vander Heiden M G, Cantley L C, Thompson C B. Understanding the Warburg effect: the metabolic requirements of cell proliferation. Science. 2009; 324(5930):1029-33. doi: 10.1126/science.1160809. PubMed PMID: 19460998; PMCID: 2849637.
16. Herst P M, Hesketh E L, Ritchie D S, Berridge M V. Glycolytic metabolism confers resistance to combined all-trans retinoic acid and arsenic trioxide-induced apoptosis in H L60rho0 cells. Leukemia research. 2008; 32(2):327-33. doi: 10.1016/j.leukres.2007.04.014. PubMed PMID: 17580091.
17. Samudio I, Fiegl M, McQueen T, Clise-Dwyer K, Andreeff M. The warburg effect in leukemia-stroma cocultures is mediated by mitochondrial uncoupling associated with uncoupling protein 2 activation. Cancer Res. 2008; 68(13):5198-205. Epub 2008/07/03. doi: 68/13/5198 [pii]
18. Herst P M, Howman R A, Neeson P J, Berridge M V, Ritchie D S. The level of glycolytic metabolism in acute myeloid leukemia blasts at diagnosis is prognostic for clinical outcome. J Leukoc Biol. 2011; 89(1):51-5. Epub 2010/10/21. doi: j1b.0710417 [pii] 10.1189/jlb.0710417. PubMed PMID: 20959411.
19. Akers L J, Fang W, Levy A G, Franklin A R, Huang P, Zweidler-McKay P A. Targeting glycolysis in leukemia: a novel inhibitor 3-BrOP in combination with rapamycin. Leukemia research. 2011; 35(6):814-20. doi:10.1016/j.leukres.2010.12.028. PubMed PMID: 21316758.
20. Chen W L, Wang J H, Zhao A H, Xu X, Wang Y H, Chen T L, Li J M, Mi J Q, Zhu Y M, Liu Y F, Wang Y Y, Jin J, Huang H, Wu D P, Li Y, Yan X J, Yan J S, Li J Y, Wang S, Huang X J, Wang B S, Chen Z, Chen S J, Jia W. A distinct glucose metabolism signature of acute myeloid leukemia with prognostic value. Blood. 2014. doi: 10.1182/blood-2014-02-554204. PubMed PMID: 25006128.
21. Mitrakas A G, Kalamida D, Koukourakis Ml. Effect of mitochondrial metabolism-interfering agents on cancer cell mitochondrial function and radio/chemosensitivity. Anticancer drugs. 2014. doi: 10.1097/CAD.0000000000000152. PubMed PMID: 25035963.
22. Aichler M, Elsner M, Ludyga N, Feuchtinger A, Zangen V, Maier S K, Balluff B, Schone C, Hierber L, Braselmann H, Meding S, Rauser S, Zischka H, Aubele M, Schmitt M, Feith M, Hauck S M, Ueffing M, Langer R, Kuster B, Zitzelsberger H, Hofler H, Walch A K. Clinical response to chemotherapy in oesophageal adenocarcinoma patients is linked to defects in mitochondria. The Journal of pathology. 2013; 230(4):410-9. doi: 10.1002/path.4199. PubMed PMID: 23592244.
23. Harper M E, Antoniou A, Villalobos-Menuey E, Russo A, Trauger R, Vendemelio M, George A, Bartholomew R, Carlo D, Shaikh A, Kupperman J, Newell E W, Bespalov I A, Wallace S S, Liu Y, Rogers J R, Gibbs G L, Leahy J L, Carnley R E, Melamede R, Newell M K. Characterization of a novel metabolic strategy used by drug-resistant tumor cells. FASEB journal: official publication of the Federation of American Societies for Experimental Biology. 2002; 16 (12):1550-7. doi: 10.1096/fj.02-0541com. PubMed PMID: 12374777.
24. Scotland S, Saland E, Skuli N, de Toni F, Boutzen H, Micklow E, Senegas I, Peyraud R, Peyriga L, Theodora F, Duman E, Martineau Y, Danet-Desnoyers G, Bono F, Rocher C, Levade T, Manenti S, Junot C, Portais J C, Alet N, Recher C, Selak M A, Carroll M, Sarry J E. Mitochondrial energetic and AKT status mediate metabolic effects and apoptosis of metformin in human leukemic cells. Leukemia. 2013; 27(11):2129-38. doi: 10.1038/leu.2013.107. PubMed PMID: 23568147.
25. Zachar Z, Marecek J, Maturo C, Gupta S, Stuart S D, Howell K, Schauble A, Lem J, Piramzadian A, Karnik S, Lee K, Rodriguez R, Shorr R, Bingham P M. Non-redox-active lipoate derivates disrupt cancer cell mitochondrial metabolism and are potent anticancer agents in vivo. Journal of molecular medicine. 2011; 89(11):1137-48. doi: 10.1007/s00109-011-0785-8. PubMed PMID: 21769686.
26. Stuart S D, Schauble A, Gupta S, Kennedy A D, Keppler B R, Bingham P M, Zachar Z. A strategically designed small molecule attacks alpha-ketoglutarate dehydrogenase in tumor cells through a redox process. Cancer & metabolism. 2014; 2(1):4. doi: 10.1186/2049-3002-2-4. PubMed PMID: 24612826; PMCID: 4108059.
27. Pardee T S, Lee K, Luddy J, Maturo C, Rodriguez R J, Isom S, Miller L O, Stadelman K M, Levitan D, Hurd D, Ellis L R, Harrelson R, Manuel M, Dralle S, Lyerly S, Powell B L. A Phase I Study of the First-in-Class Anti-Mitochondrial Metabolism Agent, C P1-613, in Patients with Advanced Hematologic Malignancies. Clinical cancer research: an official journal of the American Association for Cancer Research. 2014. doi: 10.1158/1078-0432.CCR-14-1019. PubMed PMID: 25165100.
28. Timothy S. Pardee K S, Scott Isom, Leslie R. Ellis, Dmitriy P. Berenzon, Robin Harrelson, Megan Manuel, Sarah Dralle, Susan Lyerly, Bayard L. Powell. A phase I study of the mitochondrial metabolism inhibitor C P1-613 in combination with high-dose ara-C (HDAC) and mitoxantrone for relapsed or refractory acute myeloid leukemia (AML). Journal of Clinical Oncology. 2014; 32(5s):suppl; abstr 7028.
29. Haferlach C, Dicker F, Herholz H, Schnittger S, Kern W, Haferlach T. Mutations of the T P53 gene in acute myeloid leukemia are strongly associated with a complex aberrant karyotype. Leukemia. 2008; 22(8):1539-41. doi: 10.1038/1eu.2008.143.PubMed PMID: 18528419.
30. Yin B, Kogan S C, Dickins R A, Lowe S W, Largaespada D A. Trp53 loss during in vitro selection contributes to acquired Ara-C resistance in acute myeloid leukemia. Experimental hematology. 2006; 34(5):631-41. doi: 10.1016/j.exphem.2006.01.015. PubMed PMID: 16647569.
31. Liu Y, Zhou J, White K P. RNA-seq differential expression studies: more sequence or more replication? Bioinformatics. 2014; 30(3):301-4. doi: 10.1093/bioinformatics/btt688. PubMed PMID: 24319002; PMCID: 3904521.
32. Dobin A, Davis C A, Schlesinger F, Drenkow J, Zaleski C, Jha S, Batut P, Chaisson M, Gingeras T R. STAR: ultrafast universal RNA-seq aligner. Bioinformatics. 2013; 29(1):15-21. doi: 10.1093/bioinformatics/bts635. PubMed PMID: 23104886; PMCID: 3530905.
33. Liao Y, Smyth G K, Shi W. featureCounts: an efficient general purpose program for assigning sequence reads to genomic features. Bioinformatics. 2014; 30(7):923-30. doi: 10.1093/bioinformatics/btt656. PubMed PMID: 24227677.
34. Love Ml, Huber W, Anders S. Moderated estimation of fold change and dispersion for RNA-seq data with DESeq2. Genome biology. 2014; 15(12):550. doi: 10.1186/s13059-014-0550-8. PubMed PMID: 25516281; PMCID: 4302049.
35. Ching T, Huang S, Garmine L X. Power analysis and sample size estimation for RNA-Seq differential expression. Rna. 2014; 20(11):1684-96. Doi:10.1261/rna.046011.114. PubMed PMID: 25246651; PMCID: 4201824.
36. Kramer A, Green J, Pollard J, Jr., Tugendreich S. Causal analysis approaches in Ingenuity Pathway Analysis. Bioinformatics. 2014; 30(4):523-30. doi: 10.1093/bioinformatics/btt703. PubMed PMID: 24336805; PMCID: 3928520.
37. Huang D W, Sherman B T, Tan Q, Kir J, Liu D, Bryant D, Guo Y, Stephens R, Baseler M W, Lane H C, Lempicki R A. DAVID Bioinformatics Resources: expanded annotation database and novel algorithms to better extract biology from large gene lists. Nucleic acids research. 2007; 35 (Web Server issue):W169-75. doi: 10.1093/nar/gkm415. PubMed PMID: 17576678; PMCID: 1933169.
38. Miller L D, Smeds J, George J, Vega V B, Vergara L, Planer A, Pawitan Y, Hall P, Klaar S, Liu E T, Bergh J. An expression signature for p53 status in human breast cancer predicts mutation status, transcriptional effects, and patient survival. Proceedings of the National Academy of Sciences of the United States of America. 2005; 102(38):13550-5. doi: 10.1073/pnas.0506230102. PubMed PMID: 16141321; PMCID: 1197273.
39. lvshina A V, George J, Senko O, Mow B, Putti T C, Smeds J, Lindahl T, Pawitan Y, Hall P, Nordgren H, Wong J E, Liu E T, Bergh J, Kuznetsov V A, Miller L D. Genetic reclassification of histologic grade delineates new clinical subtypes of breast cancer. Cancer research. 2006; 66 (21):10292-301. doi: 10.1158/0008-5472.CAN-05-4414. PubMed PMID: 17079448.

While certain of the preferred embodiments of the present invention have been described and specifically exemplified above, it is not intended that the invention be limited to such embodiments. It will be apparent to one skilled in the art that various changes and modifications can be made therein without departing from the scope of the present invention, as set forth in the following claims.

1. A method for detecting a collection of differentially expressed genes which indicate sensitivity to treatment with CPI-613, said method comprising:
a) obtaining a biological sample from a subject; and
b) detecting the expression levels in said biological sample of at least three genes selected from the group consisting of RP11-164H13.1, FCRLA, KLHL14, IGKV2D-29, MS4A1, DSP, COL19A1, IGKV1-8, PCDH9, TCL1A, RP11-693J15.5, IGHV3-53, IGHV3-11, FCRL2, PTPRK, BANK1, SLC16A9, and CD79.

2. The method of claim 1, wherein said three genes are RP11-164H13.1, FCRLA, and KLHL14.

3. The method of claim 1, wherein subjects that are likely to be responders or non-responders can be segregated by hierarchical cluster analysis based on expression of TCL1A, MS4A1, FCRL2, PTPRK, CD79A, BANK1, RP11-164H13.1, and RP11-693J15.5.

4. A method for detecting a differentially expressed gene which indicates a subject's sensitivity to treatment with CPI-613, said method comprising:
a) obtaining a biological sample from a subject; and
b) detecting in said biological sample the expression level of RP11-164H13.1.

5. The method of claim 1, wherein said sample is bone marrow cells, mononuclear cells from bone marrow, or peripheral blood cells.

6. A method of identifying and treating a subject likely to respond to CPI-613 treatment of AML, said method comprising:
a) obtaining a biological sample from the subject;
b) detecting in said biological sample expression levels of at least three genes selected from the group consisting of RP11-164H13.1, FCRLA, KLHL14, IGKV2D-29, MS4A1, DSP, COL19A1, IGKV1-8, PCDH9, TCL1A, RP11-693J15.5, IGHV3-53, IGHV3-11, FCRL2, PTPRK, BANK1, SLC16A9, and CD79, elevated levels of said being indicative of sensitivity to CPI-613; and
c) administering to said subject an effective amount of a treatment comprising CPI-613 and, optionally, a chemotherapeutic agent.

7. The method of claim 6, wherein said treatment comprises CPI-613, cytarabine, and mitoxantrone.

8. A method of assessing whether a subject is likely to respond to CPI-613 treatment of AML, said method comprising:
a) obtaining a biological sample from said subject;
b) contacting said sample with at least one antibody immunologically specific for at least three markers selected from the group consisting of RP11-164H13.1, FCRLA, KLHL14, IGKV2D-29, MS4A1, DSP, COL19A1, IGKV1-8, PCDH9, TCL1A, RP11-693J15.5, IGHV3-53, IGHV3-11, FCRL2, PTPRK, BANK1, SLC16A9, and CD79; and
c) detecting immune complex formation between said antibody and said marker, the presence of said immune complexes being predictive of sensitivity to CPI-613 treatment.

9. A method of detecting a collection of differentially expressed genes which indicate efficacy of CPI-613 treatment, said method comprising:
a) obtaining a biological sample from a subject; and
b) detecting the expression levels in said biological sample of at least three genes selected from the group consisting of RP11-164H13.1, FCRLA, KLHL14, IGKV2D-29, MS4A1, DSP, COL19A1, IGKV1-8, PCDH9, TCL1A, RP11-693J15.5, IGHV3-53, IGHV3-11, FCRL2, PTPRK, BANK1, SLC16A9, and CD79.

10. The method of claim 1, wherein the expression level is measured by RNA-seq.

11. The method of claim 1, wherein the expression level is measured by NanoString's nCounter technology.

12. The method of claim 8, wherein said antibodies are labeled for detection to perform immunohistochemistry, immunofluorescence imaging, or flow cytometry.

13. The method of claim 1, wherein the subject has relapsed or refractory AML.

14. The method of claim 1, wherein the subject has p53 loss or mutation.

15. A method of assessing a patient's response to CPI-613 treatment of AML, comprising
a) obtaining a biological sample from a subject following CPI-613 treatment; and
b) detecting the expression levels in said biological sample of at least three genes selected from the group consisting of RP11-164H13.1, FCRLA, KLHL14, IGKV2D-29, MS4A1, DSP, COL19A1, IGKV1-8, PCDH9, TCL1A, RP11-693J15.5, IGHV3-53, IGHV3-11, FCRL2, PTPRK, BANK1, SLC16A9, and CD79, wherein a reduction in expression levels of three or more genes is indicative of response to CPI-613 treatment of AML.
